(19) United States
(12) Patent Application Publication
(10) Pub. No.: US 2006/0019972 A1
(43) Pub. Date: Jan. 26, 2006

(54) Methods of treating cancer by inhibiting histone gene expression

(75) Inventors: Peter B. Dervan, San Marino, CA (US); Liliane A. Dickinson, San Diego, CA (US); Paramjit Arora, New York, NY (US); Ryan Burnett, San Diego, CA (US); Joel M. Gottesfeld, Del Mar, CA (US)

Correspondence Address: FOLEY & LARDNER LLP, P.O. BOX 80278, SAN DIEGO, CA, 92138-0278, US

(73) Assignee: California Institute of Technology

(21) Appl. No.: 11/116,959

(22) Filed: Apr. 27, 2005

Related U.S. Application Data
(60) Provisional application No. 60/566,017, filed on Apr. 27, 2004.
(60) Provisional application No. 60/611,687, filed on Sep. 21, 2004.

(51) Int. Cl. A61K 31/52; A61K 31/4178

(52) U.S. Cl. 514/263.2; 514/397

(57) ABSTRACT

Methods are provided for decreasing the rate or inhibiting neoplastic cell proliferation by reducing or inhibiting histone H4 gene expression or histone H4 activity. Also provided are methods of treating a patient with a neoplastic disease, and compositions useful for treating a cancer patient, including, for example, a composition containing small interfering RNA molecules that reduce or inhibit histone H4 expression in a cell or a composition containing a pyrrole-imidazole polyamide operatively linked to a chemotherapeutic molecule.

### CLAIM OF PRIORITY

This application claims benefit of priority to U.S. Provisional Application 60/566,017, filed Apr. 27, 2004 and U.S. Provisional Application 60/611,687, filed Sep. 21, 2004, each of which is fully incorporated by reference herein, including all figures, tables, references and charts.

### GOVERNMENT SUPPORT

This invention was made with government support under Grant Nos. GM57148 GM51747 and CA103711 awarded by the National Institutes of Health. The United States government may have certain rights in this invention.

### BACKGROUND OF THE INVENTION

1. Field of the Invention

The invention relates generally to therapeutic compositions and methods and more specifically to methods of treating a cancer patient by reducing or inhibiting the expression or activity histone H4 in cancer cells of the patient, and to cancer therapeutic compositions that contain an agent that reduces or inhibits the expression or activity of histone H4.

2. Background Information

Cancer remains a major cause of morbidity and mortality in humans. In addition to its impact on the cancer patient and family members, cancer inflicts a great burden on society. For example, the high cost of caring for and treating cancer patients contributes to increased cost of health insurance, which, in turn, results in a higher percent of uninsured people and, consequently, an increased economic burden on government social systems when the uninsured are sick or injured. Cancer also has a significant impact on businesses due, for example, to prolonged absences of cancer patients from work.

Methods for treating cancer have improved greatly over the years. For example, improved diagnostic methods combined with better surgical techniques allow surgeons to more confidently remove a tumor, while removing a minimal amount of normal tissue. As such, the recovery time of patients can be decreased, and psychological impact due to cosmetic trauma is reduced. However, while surgery is useful for treating patients whose tumors are localized, or have only minimally spread, for example, to local lymph nodes, it has limited usefulness for patients with metastatic disease, or with a systemic cancer such as leukemia or lymphoma.

Chemotherapy is the treatment of choice for certain types of cancers, and for treating patients whose cancers are not localized. In general, however, chemotherapeutic treatments are not specific for tumor cells, but, instead, take advantage of differences in proliferation rates of tumor cells as compared to corresponding normal cells. As a result, chemotherapy generally is associated with severe side effects, and is particularly devastating to rapidly renewing tissues such as blood forming tissues and epithelial tissues including the intestine. As such, chemotherapy often results in decreased white blood cell counts, rendering the patient susceptible to opportunistic infections, and in nausea, hair loss and other manifestations of epithelial cell damage.

More recently, agents have been identified that target specific proteins that are expressed in particular types of cancers, but not generally in all tissues. Such agents are exemplified by antibodies such as the monoclonal antibody, Herceptin® (Genentech Corp.), which is specific for a cell surface receptor that is overexpressed in breast cancer cells in about 25% to 30% of women with breast cancer. It is believed that Herceptin® acts by binding to the receptors, thereby inhibiting proliferation of the breast cancer cells. As such, Herceptin® provides a treatment that is specific for breast cancer cells, but does not substantially affect other types of cells in the body. Rituxan® (Idec-Biogen Corp.; Genentech Corp.) is another example of a monoclonal antibody that is specific, in this case for treating non-Hodgkin's lymphoma.

While specific agents such as the monoclonal antibodies described above provide a major advance in cancer treatment by allowing treatment of systemic or metastatic disease without causing systemic harm to the patient, the specificity of the agents also means that they are limited to treating one or, at best, a very few different cancers. As such, a unique agent would need to be developed for every different type of cancer. Although it is possible that, in the future, a specific therapeutic agent may be available for each specific type of cancer, the development of such therapeutic agents requires knowledge of gene or protein targets, which are expressed uniquely in the cancer cells, or at a level that is different from its expression in normal tissues. Unfortunately, only a few such target genes and proteins have been identified, and most, as discussed above, are specific to a particular type of cancer. Thus, a need exists to identify gene and/or protein targets that are differentially expressed or active in cancer cells, and particularly in a variety of different types of cancer cells, such that agents that are specific and effective for treating cancer can be developed.

### SUMMARY OF THE INVENTION

In one aspect, the invention provides a method of reducing or inhibiting proliferation of neoplastic cells. The method may include contacting neoplastic cells with an agent that binds to a histone H4 gene or to RNA that encodes histone H4. Following such contact, histone H4 activity in the cell is reduced or inhibited, which reduces or inhibits proliferation of the neoplastic cells. In some embodiments of the invention, the gene encoding histone H4 is histone H4c. In another aspect, the invention provides a method for reducing or inhibiting proliferation of neoplastic cells by contacting neoplastic cells with an agent that reduces the levels of histone H4c mRNA within the neoplastic cells.

In another aspect, the invention provides a method of screening for an agent for reducing or inhibiting proliferation of neoplastic cells. The method may include the step of measuring the ability of an agent to reduce the amount of histone H4 mRNA or histone H4 protein in a neoplastic cell. In related aspects the invention may involve measuring the ability of an agent to bind to DNA of a gene encoding histone H4. In some embodiments of the method, the gene encoding histone H4 is histone H4c.

In yet another aspect, the invention provides a method of treating a patient with a neoplastic disease; e.g. a patient with cancer. The method may include administering to such a patient an agent that binds DNA or RNA of a gene encoding histone H4 which, in turn, results in the reduction or inhibition of histone H4 activity. The reduction or inhibition of histone H4 activity thereby results in ameliorating signs of the neoplastic disease in the patient. In a related aspect the invention provides a method for treating neoplastic disease in a patient by administering an agent that reduces the levels of histone H4 in the neoplastic cells, thereby ameliorating signs of the neoplastic disease in the patient. In some embodiments of the invention, the gene encoding histone H4 that is targeted by the agent is histone H4c.

In another aspect of the invention, compositions are provided for reducing or inhibiting proliferation of neoplastic cells. The composition may include a pyrrole-imidazole polyamide operatively linked to a chemotherapeutic molecule, wherein the pyyrole and imadazole moieties are configured and arranged such that they bind DNA that contains the sequence 5′-WGGWGW-3′. As used herein in reference to a nucleic acid sequence, “W” refers to an A or a T. In a related aspect, the composition may include a DNA or RNA binding domain operatively linked to a chemotherapeutic molecule, wherein the DNA or RNA binding domain binds to DNA or RNA of a gene encoding histone H4. In certain preferred embodiments, the composition binds to DNA of the gene histone H4c. In some preferred embodiments, the chemotherapeutic molecule is an alkylator, more preferably the chemotherapeutic molecule is chlorambucil. In certain preferred embodiments, the composition is 1R-Chl. It is contemplated that a compostion of the invention may be in the form of a pharmaceutically acceptable salt or complex. The compound of the invention may be used to treat a patient with a neoplastic disease, such as cancer; that is the compound may be used to reduce or inhibit proliferation of neoplastic cells in a patient.

In yet another aspect, the invention provides a method of determining whether a neoplastic cell, or a neoplastic disease, is susceptible to treatment with an agent that reduces or inhibits histone H4 activity. The method may involve detecting the level of histone H4 in a sample of neoplastic cells and detecting the level of histone H4 in a control cell sample that corresponds to the cell type of said neoplastic cell sample; wherein at least a three-fold increase in the level of histone H4 gene expression in the neoplastic cells as compared to a level of histone H4 expression in corresponding normal cells indicates that the neoplastic cells are susceptible to treatment with an agent that reduces or inhibits histone H4 activity. The level of histone H4 gene expression may be determined, for example, by measuring levels of histone H4 protein or measuring levels of mRNA encoding histone H4. In one embodiment of the method, levels of histone H4c mRNA are measured. In related embodiments of the invention, the method of determining whether a neoplastic cell, or a neoplastic disease, is susceptible to treatment with an agent that reduces or inhibits histone H4 activity may involve contacting the neoplastic cells with an agent that reduces or inhibits histone H4 activity and evaluating proliferation of the cells.

As used herein, the term “histone H4 activity” refers to the ability of histone H4 proteins to bind DNA and form nucleosomes. For purposes of the present invention, it is considered that histone H4 activity is related, at least in part, to the level of histone H4 gene expression. As such, histone H4 activity can be reduced or inhibited by reducing or inhibiting histone H4 transcription and/or translation. Histone H4 transcription and/or translation may be reduced or inhibited using any of many methods well known in the art. For example H4 transcription and/or translation may be reduced or inhibited using a co-suppressor RNA or siRNA, respectively, specific for the target histone H4 nucleic acid molecule (e.g., histone H4c). Alternatively, H4 transcription and/or translation may be reduced or inhibited using a small organic molecule that specifically binds to, and chemically modifies, DNA or RNA of a gene encoding histone H4 (for example histone H4c). Examples of such small organic molecules include pyrrole-imidazole polyamide conjugates operatively linked to a chemotherapeutic molecule, such as 1 R-Chl. Histone H4 activity can be detected indirectly by measuring the level of mRNA encoding histone H4, or by measuring histone H4 gene transcription, in a cell.

As used herein, the term “neoplastic cells” refer to abnormal cells that grow by cellular proliferation more rapidly than normal. As such, neoplastic cells of the invention can be cells of a benign neoplasm or can be cells of a malignant neoplasm. As used herein, the term “neoplastic disease” refers to a condition in a patient which is caused by, or associated with, the presence of neoplastic cells in the patient. Cancer is one example of a neoplastic disease. In certain aspects, the neoplastic cells are cancer cells. The cancer cells can be any type of cancer, including, for example, a carcinoma, melanoma, leukemia, sarcoma or lymphoma. Exemplary cancer cells amenable to inhibition of proliferation according to a method or composition of the invention include colon carcinoma cells, hepatocellular carcinoma cells, cervical carcinoma cells, lung epidermocarcinoma cells, mammary gland adenocarcinoma cells, pancreatic carcinoma cells, prostatic carcinoma cells, osteosarcoma cells, melanoma cells, acute promyelocytic leukemia cells, acute lymphoblastic leukemia cells, hepatocancreatico adenocarcinoma cells and Burkitt's lymphoma B cells. Neoplastic cells particularly amenable to inhibition of proliferation according to a method or composition of the invention include cells that have increased levels of expression of a histone H4 gene, especially those with increased levels of histone H4c gene expression, and cell proliferation as compared to corresponding normal cellls. However, neoplastic without increased levels of histone H4 gene expression may also be affected by the methods or compositions of the invention.

The term “normal cell” is used broadly herein to refer to a non-neoplastic cell. The term “corresponding normal cell” is used herein to refer to a non-neoplastic cell that is from the same type of organism as a specified neoplastic (e.g., cancer) cell. Generally, but not necessarily, a corresponding normal cell is of the same cell type as the cell from which the cancer cell was derived (e.g., normal colon epithelial cell for colon carcinoma cell).

As disclosed herein, neoplastic cells particularly amenable to manipulation according to the methods of the invention may have increase expression levels of a particular histone gene as compared to corresponding normal cells. In certain preferred embodiments, the level of mRNA of a histone gene in neoplastic cells to be treated according to the methods or compositions of the invention is at least about 1.2-fold, at least about 1.5 fold, at least about 2-fold, at least about 2.5 fold or at least about 3-fold or greater than that of a corresponding normal cell. In certain preferred embodiments the amenable neoplastic cell has increased expression of histone H4 gene; more preferably there is an increase in the expression of histone H4c mRNA in the neoplastic cell (see, e.g., FIG. 5). In some preferred embodiments, the H4c mRNA level of a neoplastic cell amenable to the invention is at least about 1.2-fold, at least about 1.5 fold, at least about 2-fold, at least about 2.5 fold or at least more than about 3-fold greater than that of a corresponding normal cell. Increased gene expression of histone H4 in neoplastic cells can be identified, for example, by comparing the level of histone H4 gene expression in the neoplastic cells with that in corresponding normal cells (e.g., by examining the neoplastic cell and normal cell in parallel). Increased histone H4 expression in neoplastic cells also can be identified, for example, by independently (i.e., in separate experiments) examining populations of normal cells, including various types of cells (e.g., epithelial, muscle, and neuronal), and obtaining average and/or median levels of histone H4 levels (including standard deviation, standard error of the mean, or the like); the level of histone H4 in a neoplastic cell then can be compared with such known mean and/or median values to identify (or confirm) that the histone H4 expression in the neoplastic cells is increased above normal. Methods to measure gene expression are well known in the art, and any such method may be used in the invention to determine the expression level of a histone gene. For example, transcription assays, assays to measure steady-state levels of mRNA (e.g., PCR methods including semi-quantitative PCR, comparative PCR, real-time PCR; methods such as Northern Blotting, and the like) and assays to measure histone protein levels (e.g., Western-immunoblotting, enzyme-linked immunosorbent assays, radioimmunoassays, and the like) may all be used to determine the expression level of a histone gene. In related aspects, a neoplastic cell which may be ameneable to manipulation according to the methods of the invention of the invention may have an increased expression level of a gene encoding a histone other than histone H4 as compared to a corresponding normal cell; for example, a neoplastic cell may have increased levels of a gene encoding histone H2A, H2B, or H3. In certain embodiments, an amenable neoplastic cell may have an increased level of histone H3.3A or H3.3B gene expression. It is understood that a neoplastic cell with an increased expression level of a particular histone gene often will have the same total histone protein:DNA ratio as a corresponding normal cell; thus the level of total histone protein or total histone activity may be the same in an amenable neoplastic cell as a corresponding normal cell.

The terms “reduce” and “inhibit” are used together herein because it is recognized that, depending on a particular assay, it may not be possible to determine whether histone H4 activity is completely inhibited or is reduced below a level of detection for the particular assay. For example, the absence of detectable histone H4 protein by western blot analysis following treatment of neoplastic cells with an agent that reduces or inhibits histone H4 activity can indicate that histone H4 is completely absent from the cells, or can indicate that a small, but undetectable amount remains present in the cells. Regardless, however, as to whether a reduction below detectable limits or a complete inhibition of histone H4 activity has been effected following such treatment, a decrease in histone H4 activity will be measurable.

As used herein, the term “agent,” in reference to the method of the invention, means any type of molecule that can reduce or inhibit proliferation of a neoplastic cell. Molecules that may be useful as agents in the invention include, for example, peptides (or polypeptides), polynucleotides, peptidomimetics (e.g., peptide nucleic acids, PNA) and small organic molecules (e.g., polyamides). In certain embodiments, the agent reduces the expression of a histone gene, more preferably the expression of a histone H4 gene, more preferably the expression of histone H4c. In some embodiments, the agent may comprise a pyrrole-imidazole polyamide moiety that is operatively linked to a chemotherapeutic molecule. An example of one preferred agent of the method is 1R-Chl (FIG. 1). In other embodiments, the agent may be a nucleic acid molecule, such as siRNA, that inhibits the expression of a histone gene.

The terms “polynucleotide” and “nucleic acid molecule” are used broadly herein to refer to a sequence of two or more deoxyribonucleotides, ribonucleotides or analogs thereof that are linked together by a phosphodiester bond or other known linkages. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. The terms also are used herein to include naturally occurring nucleic acid molecules, which can be isolated from a cell using recombinant DNA methods, as well as synthetic molecules, which can be prepared, for example, by methods of chemical synthesis or by enzymatic methods such as by PCR. The term “recombinant” is used herein to refer to a nucleic acid molecule that is manipulated outside of a cell, including, for example, a polynucleotide encoding an siRNA specific for a histone H4 gene operatively linked to a promoter.

The term “operatively linked”, “in operative linkage”, “linked” or “operatively associated” is used herein to refer to two or more molecules that, when joined together, act in concert. For example, when used in reference to a transcriptional regulatory element (e.g., a promoter) and a second nucleotide sequence (e.g., a polynucleotide encoding an siRNA), the term “operatively linked” means that the regulatory element is positioned with respect to the second nucleotide sequence such that the regulatory element functions to effect transcription of the second nucleotide sequence (e.g., a promoter effects transcription of an operatively linked coding sequence). Also, with respect to a polynucleotide encoding an siRNA (or co-suppressor RNA), reference to the first oligonucleotide being in operative linkage to the second oligonucleotide means that an RNA molecule comprising the first and second oligonucleotides can form a hairpin structure having siRNA or co-suppressor RNA activity, or that two RNA molecules are expressed, which, in a cell, hybridize to form an siRNA. Where the first and second oligonucleotide are expressed as a single unit, they can be linked by a spacer nucleotide sequence that provides sufficient spacing between the first and second oligonucleotides such that self-hybridization of a single stranded form of the nucleic acid molecule (e.g., RNA) is not constrained, and a hairpin can form. Further, an agent such as a nucleic acid molecule (e.g., antisense molecule) or small organic molecule can be operatively associated to a second molecule of interest, for example, a detectable label to identify intracellular localization of the agent or a chemotherapeutic molecule, to form a conjugate, wherein each component of the conjugate exhibits an effect characteristic of the individual component, alone. As exemplified herein, a PI-polyamide-chlorambucil conjugate exhibited histone H4 target specificity (due to the PI-polyamide component) and DNA alkylating activity (due to the chlorambucil component).

The term “chemotherapeutic molecule” as used herein, refers to a chemical, or a chemical moiety, that alters the morphology or growth characteristics of neoplastic cells in culture or in vivo. Preferable chemotherapeutic molecules reduce the aberrant proliferation of neoplastic cells. Examples of chemotherapeutic molecules include, but are not limited to DNA alkylators, topoisomerase inhibitors or histone deacetylase inhibitors. It is understood that a chemotherapeutic molecule of the invention may be conjugated or linked to another functional moiety such as a nucleic acid binding domain. Particularly preferable chemotherapeutic molecules can be conjugated or linked to a separate moiety, such as a DNA or RNA binding moiety, to increase the specificity of the chemotherapeutic molecules and/or decrease the toxicity or side-effects of the agent. Particularly useful chemotherapeutic molecules of the invention are DNA alkylators (e.g. chlorambucil).

As used herein, the term “alkylator” means a compound that reacts with and adds an alkyl group to another molecule. In preferred embodiments, the alkylator is reactive with DNA at about 37 degrees Celsius, the alkylator is substantially inert in aqueous media, and/or the alkylator is present in a buffer and the alkylator is non-reactive with the buffer. Non-limiting examples of alkylators that may be used in the invention include cyclophosphamide, nitrosoureas, mitozolomide, anthramycin, bromoacetyl, a nitrogen mustard, clorambucil, a derivative of chlorambucil (such as a Bis(dichloroethylamino)benzene derivative), seco-CBI, mitomycin, initomycin C, or (+)-CC-1065. Seco-CBI is a precursor to 1,2,9,9a-tetrahydrocyclopropa[1,2-c]benz[1,2-e]indol-4-one (CBI), (Boger, D. L. et al. Bioorgan. Med. Chem. 1995, 3, 1429-1453; and Boger, D. L. and Johnson, D. S. Angew. Chem., Int. Ed. Engl. 1996, 35, 1438-1474) an analogue of the natural product (+)-CC-1065. CBI shows increased reactivity to DNA as well as increased stability to solvolysis (Boger, D. L. and Munk, S. A. J. Am. Chem. Soc. 1992, 114, 5487-5496). The seco agents readily close to the cyclopropane forms and have equivalent reactivity as compared to CBI, but have been shown to have longer shelf lives (Boger, D. L. et al. Bioorg. Med. Chem. Lett. 1991, 1, 55-58).

As used herein the term “about” refers to the indicated value ±10%.

### BRIEF DESCRIPTION OF THE DRAWINGS

FIG. 1 shows the chemical structure of polyamides 1R and 1S and the structures of the bodipy and Chl conjugates. Polyamide structures are represented schematically, with filled circles representing Im rings; open circles representing Py rings; diamonds representing β-alanine; the curved line representing R or S-2,4-diaminobutyric acid; and the semicircle with plus sign representing dimethylaminopropylamine.

FIG. 2. Shows the effect of polyamide 1R-Chl on the morphology and growth of SW620 as evaluated by fluorescence activated cell-sorting analysis. The SW620 cells were either untreated or were treated with 0.5 μM 1R-Chl or 0.5 μM 1S-Chl, for 48 h prior to staining with propidium iodide (50 μg/ml). Cell numbers versus propidium staining are plotted and the percentages of cells in G0/G1, S, and G2/M phases of the cell cycle are indicated.

FIG. 3 shows the effects of 1R-Chl or Chl on the viability and cell number of cultures of SW620 cells. Viability was measured with an ATP metabolic assay (ApoSensor).

FIG. 4 shows the effects of scrambled siRNA, siRNA to histone H4c and 1R-Chl on the growth of SW620 cells.

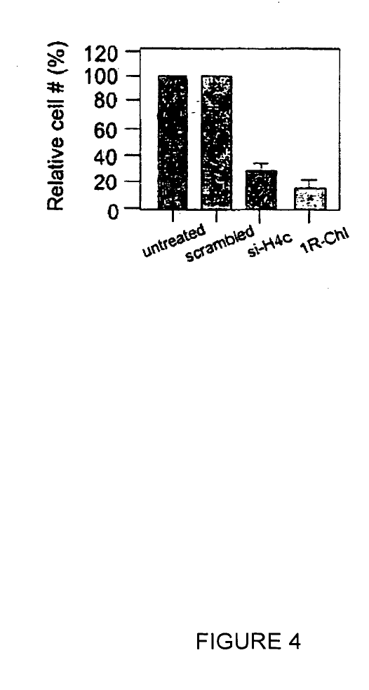

FIG. 5 shows the relative levels of mRNA abundance (RMA values) from Affymetrix GeneChip U133A analysis for each of the human histone H4 genes. Data are shown for SW620 cells, SW620 cells treated with 1R-Chl (denoted 48R-Chl), MT2 cells, normal kidney and peripheral blood mononuclear cells (PBL).

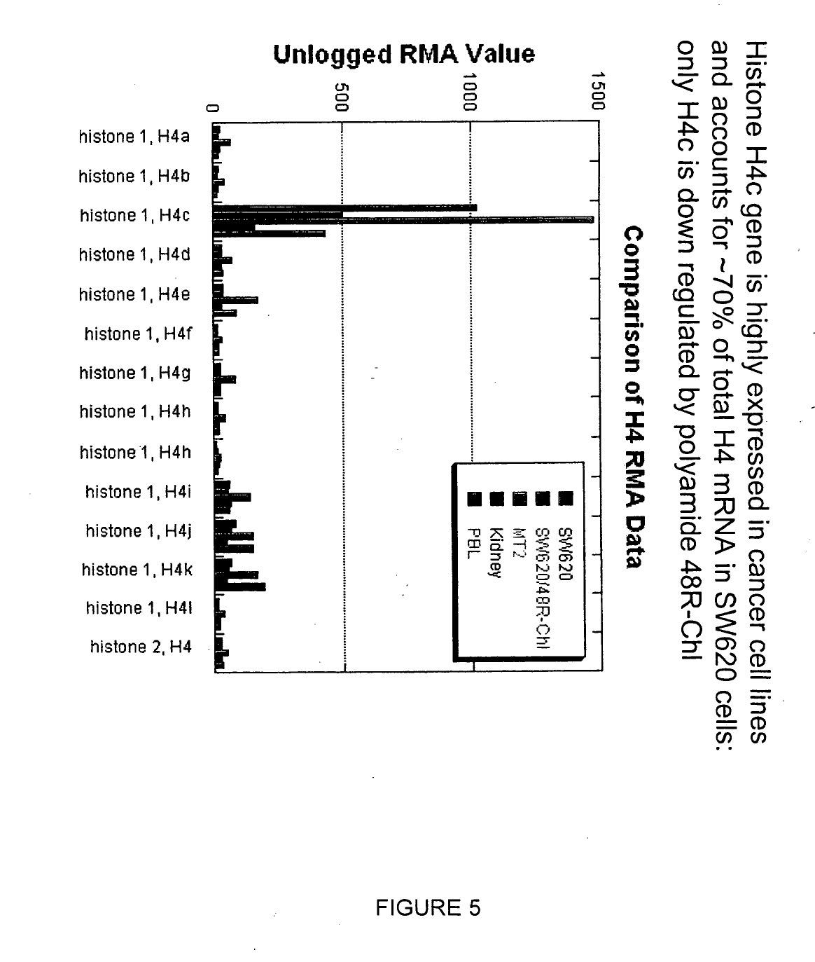

FIG. 6 shows the effects of polyamides 1R-Chl and 1S-Chl on the growth and viability of (A) Hep3B cells; (B) HeLa cells; (C) K562 lymphoid cells.

FIG. 7 shows the effect of polyamide 1R-Chl on tumor growth in athymic nude/nu mice. In experiment 1, tumor weight at 28 days post injection of 1×10⁷SW620 cells is indicated as mean, range of observed values, and standard deviation (vertical line) for each group of 5 treated or untreated mice. In experiment 2, tumor volumes were determined 18 days post injection of SW620 cells, and at 15 days post treatment (day 33) with 120 nmole of 1R-Chl or 1S-Chl, as described in the text. Mean and standard deviations for four mice are indicated.

### DETAILED DESCRIPTION OF THE INVENTION

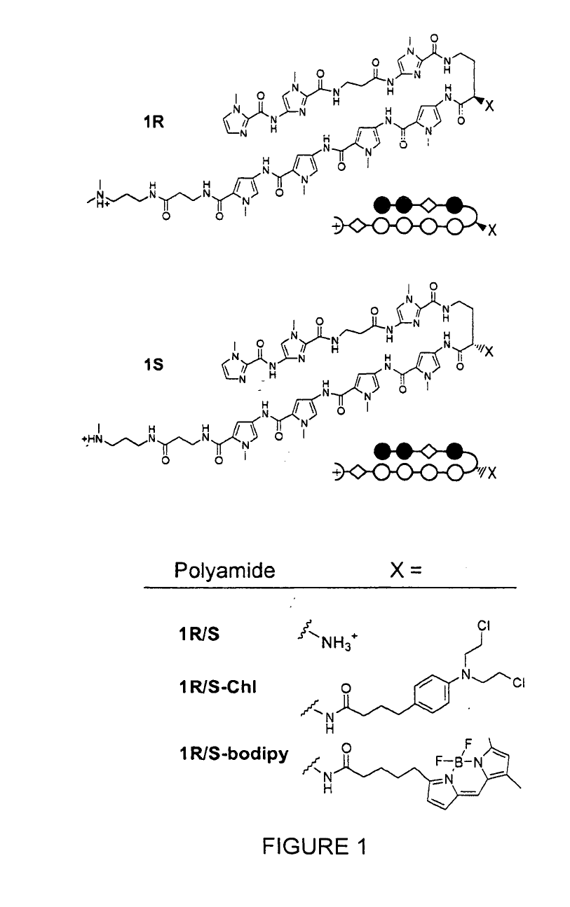

The present invention is based, in part, on the discovery that pyrrole-imidazole (PI) polyamide-DNA alkylator (chlorambucil) conjugates affect the morphology and growth characteristics of human colon carcinoma cell lines. In particular, 1R-Chl (FIG. 1; also referred to as “48R-CHL”) caused cells to arrest in the G2/M stage of the cell cycle, without any apparent cytotoxicity. Microarray analysis indicated that only one gene, histone H4c, was significantly down regulated by the 1R-Chl, demonstrating the selectivity of the compound. Further microarray studies indicated that H4c was the most highly expressed histone H4 gene in SW620 cells, accounting for approximately 70% of total H4 mRNA. Down regulation of H4c mRNA by siRNA yielded the same cellular response as 1R-Chl, providing target validation. The compound 1R-Chl also blocked tumorigenicity of metastatic colon carcinoma cells when administered in vivo by intravenous injection in immunocompromised mice. In vivo studies also showed that therapeutically effective amounts of 1R-Chl exhibited favorable pharmacokinetic properties and caused no apparant toxicity. Histone H4, and in particular the gene histone H4c, therefore offer a new target for reducing or inhibiting proliferation of neoplastic cells.

Accordingly, methods of reducing or inhibiting proliferation of neoplastic cells are provided in the present invention. Generally the method involves contacting the neoplastic cells with an agent that causes a reduction in the levels or activity of histone H4 in the neoplastic cells. Thus, the methods of the invention utilize an agent that reduces or inhibits histone H4 activity in the neoplastic (e.g., cancer) cells such that, upon contact with the cells, histone H4 activity is reduced or inhibited. In certain embodiments, the agent reduces histone H4 protein levels or activity by causing a reduction in the levels of mRNA encoding histone H4 within the neoplastic cell. Such a reduction of mRNA could, for example, occur as a result of inhibition or impairment of the transcription of the gene encoding the mRNA of interest; or by posttranscriptional effects such as the degradation of mRNA transcripts or the impairment of translation. The reduction of histone H4 may result from the reduction of mRNA levels of any of the family of genes that code for histone H4. For example the human genome contains 14 genes that encode the same histone H4 protein. In preferred embodiments, however, the mRNA level of histone H4c mRNA is reduced by the method. Thus, in one embodiment, a method of reducing or inhibiting proliferation of neoplastic cells is provided which involves contacting the neoplastic cells with an agent that reduces histone H4c mRNA levels in the neoplastic cells. The reduction in histone H4c mRNA in turn results in a reduction or inhibition proliferation of the neoplastic cells. In some preferred embodiments, the agent of the invention specifically reduces H4c mRNA; that is, histone H4c expression is the predominant gene affected by the agent.

In related embodiments, the invention involves a method of reducing or inhibiting proliferation of neoplastic cells that have elevated levels of gene expression of a histone H4 gene. As such, in one embodiment the method may involve contacting the neoplastic cells with an agent that reduces histone H4 mRNA or histone H4 protein levels, wherein, prior to the contacting step, the neoplastic cells have expression levels of a histone H4 gene that are at least three-fold higher than the level in corresponding normal cells. In certain preferred embodiments, the histone H4 gene is histone h4c. In certain embodiments the reduction in histone H4 mRNA or protein by methods or compositions of the invention may be ineffective in reducing or inhibiting proliferation of neoplastic cells that do not have increased expression levels of a histone H4 gene.

In certain embodiments, the method may include contacting neoplastic cells with an agent that binds to a histone H4 gene or RNA encoding histone H4. As such, the agent of the method may bind to DNA or RNA of a histone H4 gene. As used herein the term “histone H4 gene” includes any gene that contains encodes histone H4 protein, including any of the fourteen known human histone H4 genes. The term “binding” as used herein in the context of an agent binding to DNA or RNA broadly refers to any chemical interaction between the agent and the particular DNA or RNA of interest. One example of binding is hybridization, such as that which occurs between nucleic acid molecules used an agent (e.g. oligonucleotides, antisense nucleotides, siRNA, RNAi etc.; see below for further discussion) and DNA or RNA in the cell. Another example of binding that may be applicable to the invention includes interactions between Pyrrole-Imidazole polyamide molecules and target DNA (see below and see also U.S. Pat. No. 6,559,125). Preferably, the binding of an agent to DNA or RNA of a gene encoding histone H4 in a neoplastic cell results, either directly or indirectly, in a reduction or inhibition of histone H4 activity in the cell, which in turn results in a reduction or inhibition of proliferation of the neoplastic cell. DNA of a gene encoding histone H4 may be any sequence of nucleic acids in the genome of the neoplasic cell that is part of a gene encoding histone H4.Such a DNA sequence may be found in the coding region of the gene, the promoter region of the gene, or an exon of the gene. Preferably the binding of the agent to the DNA results, either directly or indirectly, by influencing the expression of H4 protein. Agents binding to regions of the genome near a histone H4 gene, such as an enhancer or suppressor region, could also influence the expression of histone H4 protein; therefore agents with such properties are also an object of the invention. RNA of a gene encoding histone H4 refers a sequence of RNA that encodes histone H4. As such, RNA of a gene encoding histone H4 may include mRNA transcribed from any of the 14 human genes that encode histone H4 protein. In certain embodiments, the RNA encoding histone H4 is mRNA of the histone H4c gene. Preferably agents that bind to histone H4 mRNA act to prevent translation of the mRNA. In preferred embodiments, agents of the invention bind to DNA or RNA of histone H4c. In certain preferred embodiments, a particularly preferable agent of the invention binds to DNA which contain the sequence 5′-WGGWGW-3′. In certain preferred embodiments, the agent binds to the DNA or RNA of interest in the target cell and causes a chemical modification of the DNA or RNA. Examples of such chemical modifications include, but are not limited to, alkylation or degradation of the DNA or RNA.

The invention also includes compositions for reducing or inhibiting proliferation of neoplastic cells by reducing or inhibiting the expression of a histone gene. Preferable compositions reduce or inhibit histone H4 mRNA levels, more preferably histone H4c mRNA levels, in a neoplastic cell. The compositions may have a DNA or RNA binding domain operatively linked to a chemotherapeutic molecule, wherein the DNA or RNA binding domain binds to DNA or RNA of a gene encoding histone H4. A composition useful for inhibiting histone H4 expression, and therefore proliferation of neoplastic cells, can be any type of agent, including, for example, a peptide (or polypeptide), a nucleic acid molecule (DNA or RNA), a peptidomimetic (e.g., a peptide nucleic acid, PNA), or a small organic molecule (e.g., a polyamide). In some preferred embodiments, a composition or agent of the invention reduces histone H4 activity by reducing the level of H4c mRNA.

In certain aspects, the invention provides compounds combining sequence-specific recognition of DNA with alkylation. Thus agents of the invention which are useful for reducing or inhibiting proliferation of neoplastic cells include compounds which have an alkylator combined to a DNA binding region that is capable of specifically binding DNA of a gene encoding histone H4.

DNA alkylators were among the first anti-cancer drugs developed and are the most commonly used agents in cancer chemotherapy. (Zewail-Foote, et al., Anticancer Drug Des 14, 1-9 (1999)). Alkylators induce cross-linking of DNA strands, abnormal base pairing, or DNA strand breaks, thus blocking cells in the G2/M phase of the cell cycle, thereby preventing cancer cell proliferation. Since conventional alkylators modify DNA at numerous sites in the genome, considerable effort has been expended to devise more sequence-specific alkylators, in the hope that increasing DNA sequence specificity will decrease the unwanted side effects of nonspecific alkylators, while retaining the ability of the compound to kill cancer cells. Two approaches that have been taken are development of DNA alkylators with some degree of DNA sequence specificity, such as the duocarmycins and pyrrolobenzodiazepines (Boger, et al., Bioorg Med Chem Lett 10, 495-8 (2000); Gregson, et al., J Med Chem 44, 737-48 (2001)), and linking existing alkylators, such as chlorambucil or the duocarmycins with more sequence-specific DNA-binding small molecules. (Wurtz, et al., Chem. & Biol. 7, 153-161 (2000); Shinohara, et al., J. Am. Chem. Soc. (2004)).

A number of small molecule-alkylator conjugates have been explored as potential cancer therapeutics. One agent that has been used in cancer chemotherapy is the bifunctional alkylator tallimustine, a synthetic derivative of tri-pyrrole distamycin A, in which the NH₂-terminal formyl group is substituted by the benzoyl mustard chlorambucil (Chl). Studies in SCID mice using human leukemic cell lines demonstrated that tallimustine is effective in prolonging survival, and in producing some cures in mice. Following these observations, a phase I-II study of tallimustine in acute leukemia and other advanced cancers was initiated. (Weiss, G. R. et al. Clin Cancer Res 4, 53-9 (1998). However, tallimustine is dose-limited by myelosuppression because of a lack of specificity.

The pyrrole-imidazole polyamides are a class of small molecules that can be designed to bind predetermined DNA sequences (See, e.g., Dervan, P. B., Bioorgan. Med. Chem. (2001) 9: 2215-2235; Dervan, et al., Curr. Opin. Struct. Biol. (2003) 13: 284-299; Marques et al., J. Am. Chem. Soc. (2004) 126: 10339-10349; Renneberg et al., J. Am. Chem. Soc. (2003) 125:5707-5716; Foister et al., Bioorg. Med. Chem. II (2003) 4333-4340; Doss et al., Chemistry & Biodiversity (2004) 1:886-899; Briehn et al., Chem. Eur. J. (2003) 9:2110-2122; and U.S. application Ser. No. 11/038,506, filed Jan. 18, 2005). These molecules bind their target sites in genomic DNA with affinities comparable to natural DNA-binding transcription factors. (Dudouet, B. et al. (2003) Chem Biol 10: 859-67). Additionally, polyamide-alkylator conjugates can deliver an alkylation warhead to pre-determined sites in human genomic DNA, in the cell nucleus. (Wurtz, et al. (2000) Chem. & Biol. 7: 153-161). Inhibition of transcription in vitro (Oyoshi, et al. (2003) J Am Chem Soc 125: 4752-4) and luciferase expression in mammalian cell culture transfection experiments has been obtained with polyamide-alkylator (duocarmycin DU86) conjugates. (Shinohara, K. et al. (2004) J. Am. Chem. Soc.).

As used herein, the term “polyamide” refers to polymers of amino acids covalently linked by amide bonds (see, for example U.S. Ser. No. 08/607,078, PCT/US97/03332, U.S. Ser. No. 08/837,524, U.S. Ser. No. 08/853,525, PCT/US97/12733, U.S. Ser. No. 08/853,522, PCT/US97/12722, PCT/US98/06997, PCT/US98/02444, PCT/US98/02684, PCT/US98/01006, PCT/US98/03829, and PCT/US98/0714 all of which are incorporated herein by reference in their entirety, including any drawings). Preferably, the amino acids used to form these polymers include N-methylpyrrole (Py) and N-methylimidazole (Im). Polyamides containing pyrrole (Py), and imidazole (Im) amino acids are synthetic ligands that have an affinity and specificity for DNA comparable to naturally occurring DNA binding proteins. (See, e.g., Trauger, J. W., Baird, E. E. & Dervan, P. B. (1996), Nature 382, 559-561; Swalley, S. E., Baird, E. E. & Dervan, P. B. (1997), J. Am. Chem. Soc. 119, 6953-6961; Turner, J. M., Baird, E. E. & Dervan, P. B. (1997), J. Am. Chem. Soc. 119, 7636-7644; Trauger, J. W., Baird, E. E. & Dervan, P. B. (1998), Angewandte Chemie-International Edition 37, 1421-1423; and Dervan, P. B. & Burli, R. W. (1999), Current Opinion in Chemical Biology 3, 688-693).

The particular order of amino acids in such polyamides, and their pairing in dimeric, antiparallel complexes formed by association of two polyamide polymers, determines the sequence of nucleotides in dsDNA with which the polymers preferably associate. The development of pairing rules for minor groove binding polyamides derived from N-methylpyrrole (Py) and N-methylimidazole (Im) amino acids provided a useful code to control target nucleotide base pair sequence specificity. Specifically, an Im/Py pair in adjacent polymers was found to distinguish G●C from C●G and both of these from A●T or T●A base pairs. A Py/Py pair was found to specify A●T from G●C but could not distinguish A●T from T●A. More recently, it has been discovered that inclusion of a new aromatic amino acid, 3-hydroxy-N-methylpyrrole (Hp) (made by replacing a single hydrogen atom in Py with a hydroxy group), in a polyamide and paired opposite Py enables A●T to be discriminated from T●A by an order of magnitude. Utilizing Hp together with Py and Im in polyamides provides a code to distinguish all four Watson-Crick base pairs (i.e., A●T, T●A, G●C, and C●G) in the minor groove of dsDNA as follows:

Pairing Code for Minor Groove Recognition

| Pair | G · C | C · G | T · A | A · T |
|---|---|---|---|---|
| Im/Py | + | − | − | − |
| Py/Im | − | + | − | − |
| Hp/Py | − | − | + | − |
| Py/Hp | − | − | − | + |

Favored (+),
disfavored (−)

As discussed above, a number of different polyamide motifs have been reported in the literature, including “hairpins,” “H-pins,” “overlapped,” “slipped,” and “extended” polyamide motifs. Specifically, hairpin polyamides are those wherein the carboxy terminus of one amino acid polymer is linked via a linker molecule, typically aminobutyric acid or a derivative thereof to the amino terminus of the second polymer portion of the polyamide. Indeed, the linker amino acid γ-aminobutyric acid (γ), when used to connect first and second polyamide polymer portions, or polyamide subunits, C→N in a “hairpin motif,” enables construction of polyamides that bind to predetermined target sites in dsDNA with more than 100-fold enhanced affinity relative to unlinked polyamide subunits. (See, for example, Turner, et al. (1997), J. Am. Chem. Soc., 119: 7636-7644; Trauger, et al. (1997), Angew. Chemie. Int. Ed. Eng., 37:1421-1423; Turner, et al. (1998), J. Am. Chem. Soc., 120: 6219-6226; and Trauger et al. (1998), J. Am. Chem. Soc., 120:3534-3535). Paired β-alanine residues (β/β), restore the curvature of the dimer for recognition of larger binding sites and in addition, code for AT/TA base pairs. (Trauger, J. W., Baird, E. E., Mrksich, M. & Dervan, P. B. (1996), J. Am. Chem. Soc. 118, 6160-6166; Swalley, S. E., Baird, E. E. & Dervan, P. B. (1997), Chem.-Eur. J. 3, 1600-1607; and Trauger, J. W., Baird, E. E. & Dervan, P. B. (1998), J. Am. Chem. Soc. 120, 3534-3535. Eight ring hairpin polyamides can bind a 6 base pair match sequence at subnanomolar concentrations with good sensitivity to mismatch sequences. Dervan, P. B. et al. (1999), Curr. Opin. Chem. Biol. 3: 688-693. Moreover, eight-ring hairpin polyamides (comprised of two four amino acid polymer portions linked C→N) have been found to regulate transcription and permeate a variety of cell types in culture (See Gottesfield, J. M. et al. (1997), Nature, 387:202-205 (1997).

An H-pin polyamide motif, i.e., wherein two paired, antiparallel polyamide subunits are linked by a linker covalently attached to an internal polyamide pair, have also been reported. Another polyamide motif that can be formed between linked or unlinked polyamide subunits is an “extended” motif, wherein one of the polyamide subunits comprises more amino acids than the other, and thus has a single-stranded region. See U.S. Ser. No. 08/607,078. In contrast, an “overlapped” polyamide is one wherein the antiparallel polyamide subunits completely overlap, whereas in a “slipped” binding motif, the two subunits overlap only partially, with the C-terminal portions not associating with the N-terminal regions of the other subunit. See U.S. Ser. No. 08/607,078.

Hairpin polyamide-dye conjugates enter the nucleus of cultured SW620 cancer cells and other cell lines in culture. (Best et al. (2003) Proc. Natl. Acad. Sci. USA 100, 12063-68). Polyamide-chlorambucil conjugates blocked transcription by mammalian RNA polymerase II when the conjugates were targeted to the coding regions of genes, both in vitro and in cell culture, similar to the results reported for polyamide-duocarmycin conjugates. (Shinohara K et al. (2003) J. Am. Chem. Soc.; Oyoshi T et al., (2003) J Am Chem Soc 125, 4752-4). Polyamides, such as PI-polyamides, which are useful alone, or as conjugates, can be prepared as described (see U.S. Pat. No. 6,559,125, which is incorporated herein by reference).

A small organic molecule useful for inhibiting proliferation of neoplastic cells according to a method of the invention is exemplified by a polyamide such as a pyrrole-imidazole polyamide. In one aspect, the pyrrole-imidazole polyamide (PI polyamide) comprises a conjugate, which include a chemotherapeutic molecule operatively linked to the pyrrole-imidazole polyamide. Such a conjugate is exemplified by a pyrrole-imidazole polyamide having a DNA alkylator (e.g., chlorambucil) operatively linked thereto. Examples of conjugated pyrrole-imidazole polyamides as well as methods for designing conjugated pyrrole-imidazole polyamides that could be useful in the present invention are disclosed in U.S. Pat. No. 6,559,125, which is incorporated herein by reference.

As disclosed herein, the pyrrole-imidazole polyamide-chlorambucil conjugate 1R-Chl alters cell morphology and causes arrest of colon carcinoma cells at the G2/M stage of the cell cycle, without any apparent cytotoxicity (see Example 1). Microarray analysis revealed that only one gene, histone H4c, is significantly down-regulated due to exposure of the cells to 1R-Chl even though potential binding sites for the polyamide (i.e. 5′-WGGWGW-3′) are present thousands of times in the human genome. RT-PCR and western blot experiments confirmed that histone H4 mRNA and protein were down regulated.

The chemical structure of 1R, 1S and biodipy and Chl conjugates (ImIm-β-Im-(R/S-2,4-Daba^{Bodipy/chl})-PyPyPyPy-β-Dp, where Py is pyrrole, Im is imidazole, β is β-alanine, Dp is dimethylaminopropylamine, and Daba is either R- or S-2,4-diaminobutyric acid) are shown in FIG. 1. A variety of modifications could be made to the 1R-Chl molecule and be within the scope of the invention. For example, the C-terminal beta-alanine residue can be omitted. Likewise the charged C-terminal group (Dp) can be omitted or substituted with another group. One of skill in the art also will recognize that substitutions to the N-methyl positions on the various heterocycles can be made that will not disturbing binding. Likewise, substitutions to the various heterocycles can be included such as those described, for example, in copending U.S. patent application Ser. No. 10/794,584, and those described, for example in Marques, M. A. et al. Helvetica Chimica Acta 85 (12): 4485-4517 (2002). For example, based on the pairing rules for DNA recognitions with pyrrole-imidazole polyamides, one could design other pyrrole-imidazole polyamides that target the same nucleic acid sequence as 1R-Chl, i.e., 5′-WGGWGW-3′. Examples such polyamides include ImImPyIm-(R-2,4-Daba^{Chl})-PyPyPyPy-β-Dp, ImImPyIm-(R-2,4-Daba^{Chl})-PyβPyPy-β-Dp, and ImImβIm-(R-2,4-Daba^{Chl})-PyβPyPy-β-Dp, but other polyamides could be designed to target this sequence, and these examples are not limiting. However, the sequence 5′-AGGAGA-3′ is of the form 5′-WGGWGW-3′, but is not bound by 1R-Chl; therefore this sequence likely does not represent a desirable target sequence for new polyamides.

Pyrrole-imidazole polyamide agents of the invention that differ from R1-Chl can be designed by targeting sequences of the histone H4c gene overlapping the sequence targeted by polyamide 1R-Chl (also known as 48R-Chl), and alkylate adjacent purine residues in the minor groove, similar to 1R-Chl. The region surrounding the 1R-Chl binding and alkylation site in the histone H4c gene is 5′- . . . ATGAGGAGACTCGAGGTGTGCTTAAGGTTTTCTT . . . -3′, where the binding site for 1R-Chl is underlined and the guanine that base pairs with the italicized cytosine is the site of alkylation (taken from GenBank accession number NM_{—}003542). Examples of polyamides that would bind and alkylate DNA in this region include:

ImPyImIm-(R-2,4-Daba^{Chl})-PyPyPyPy-β-Dp, ImβImIm-(R-2,4-Daba^{Chl})-
PyPyPyPy-β-Dp, ImβImIm-(R-2,4-Daba^{Chl})-PyPyβPy-β-Dp, and
ImPyImIm-(R-2,4-Daba^{Chl})-PyPyβPy-β-Dp for the sequence 5′-
TGAGGA-3′.
ImPyImPy-(R-2,4-Daba^{Chl})-ImPyPyPy-β-Dp, ImβImPy-(R-2,4-Daba^{Chl})-
ImPyPyPy-β-Dp, ImβImPy-(R-2,4-Daba^{Chl})-ImPyβPy-β-Dp, and
ImPyImPy-(R-2,4-Daba^{Chl})-ImPyβPy-β-Dp for the sequence 5′-TGTGCT-
3′. (This sequence overlaps the binding site for 1R-Chl(5′-AGGTGT-
3′)).
ImImPyPy-(R-2,4-Daba^{Chl})-PyPyPyPy-β-Dp for the sequence 5′-
AGGTTT-3′.

One polyamide, ImImPyPy-(R-2,4-Daba^{Chl})-ImPyPyPy-β-Dp, down regulates H4c gene transcription and causes growth and cell cycle arrest in the human chronic myelogenous leukemia cell line K562. This compound, has been found to alkylate multiple sites in the H4c gene of the form 5′-WGGWCN-3′ in vitro, and likely works in cells by the same mechanism as 1R-Chl.

As a result of their DNA binding properties polyamides deliver reactive moieties for covalent reaction at specific DNA sequences of a histone H4 gene and thereby inhibit DNA-protein interactions. This site specific alkylation of DNA enables regulation of gene expression. In addition to competing with transcription factors or promoters, the conjugates of the present invention could be used to target a histone H4 gene's coding region. This allows the use of synthetic chemistry to create a new class of gene specific “knockout” reagents which can reduce or inhibit proliferation of neoplasitic cells by inhibiting the levels of histone H4 mRNA.

Thus, in certain aspects the invention provides compositions for reducing or inhibiting proliferation of neoplastic cells, wherein the compositions have a pyrrole-imidazole polyamide region operatively linked to a chemotherapeutic molecule wherein the pyyrole and imadazole moieties are configured and arranged such that they bind DNA of a gene encoding histone H4. In certain preferred embodiments, the pyrrole-imidazole region is can bind DNA that contains the sequence 5′-WGGWGW-3′.

As disclosed herein, nucleic acid molecules such as antisense molecules, ribozymes, triplexing agents, siRNA, and co-suppressor RNA molecules may be used as the histone DNA or RNA binding component of an agent of the invention. A nucleic acid molecule agent is exemplified by a nucleic acid that reduces or inhibits the mRNA of a histone H4 gene, thereby decreasing histone H4 levels and, consequently, histone H4 activity in the neoplastic cells (e.g., a co-suppressor RNA or a triplexing agent). A nucleic acid molecule agent also can reduce or inhibit translation of mRNA expressed from the histone H4 gene, whereby histone H4 levels and, therefore, histone H4 activity is reduced or inhibited in the neoplastic cell (e.g., a small interfering RNA (siRNA), a co-suppressor RNA, a triplexing nucleic acid molecule, an antisense molecule, or a ribozyme). In one aspect, the nucleic acid molecule agent is an siRNA, an example of which has a nucleotide sequence as set forth in SEQ ID NO:7. A nucleic acid molecule useful according to the present methods also can act by reducing or inhibiting the expression of a transcription factor that regulates expression of the histone H4 gene.

An antisense molecule, for example, can bind to a histone H4 mRNA to form a double stranded molecule that cannot be translated in a cell. Antisense oligonucleotides of about 15 to 25 nucleotides are preferred since they are easily synthesized and can hybridize specifically with a target sequence, although longer antisense molecules can be used. Where the antisense molecule is contacted directly with a target cell, it can be operatively associated with a chemically reactive group such as iron-linked EDTA, which cleaves a target RNA at the site of hybridization. A triplexing agent, in comparison, can stall transcription (Maher et al. (1991), Antisense Res. Devel. 1:227; Helene (1991), Anticancer Drug Design 6:569).

As exemplified herein, siRNA molecules can be particularly useful for reducing or inhibiting translation of histone H4 mRNA and, therefore, histone H4 activity in neoplastic cells. Silencing gene expression at the mRNA level is referred to as RNA interference. siRNA molecules are double stranded RNA (“dsRNA” ) that effect post-transcriptional gene silencing, a naturally occurring phenomenon in plants and fungi (Cogoni and Macino (1999) Curr. Opin. Microbiol. 6:657-62). When introduced into worms, flies, or early mouse embryos, siRNA induces a cellular response that degrades the mRNA that shares the same sequence with one strand of the dsRNA (Fire, (1999) Trends Genet. 9:358-363). In some systems, a few copies of the siRNA can induce total degradation of target mRNAs (Fire et al. (1998) Nature 6669:806-811). siRNA can be successfully applied to silence almost any sequence in mRNAs (Caplen et al. (2001) Proc. Natl. Acad. Sci. USA 17:9742-9747); (Caplen et al. (2000) Gene 1-2:95-105; Oates et al. (2000) Devel. Biol. 1:20-8). By using a short, 21 to 23 nucleotide dsRNA, Elbashir et al. and other researchers showed that small interfering RNA (siRNA) could reduce or knock down specific gene expression without causing a global shut-down (Caplen et al. (2001) Proc. Natl. Acad. Sci. USA 17:9742-7; Elbashir et al. (2001), Nature 6836:494-8).

A nucleic acid molecule agent of the invention is exemplified by the siRNA to histone H4c, SEQ ID NO:7, that effectively reduces histone H4 activity in colon carcinoma cells and inhibites proliferation of the cells (see Example 2). siRNA useful for the present methods can be obtained, for example, using an in vitro transcription system or can be synthesized chemically, and can be contacted with cells (or administered to a subject) as RNA molecules. siRNA also can be expressed from an encoding nucleic acid molecule, which can be contacted with neoplastic cells (or administered to a subject), wherein the siRNA is expressed in the cells. siRNA molecules can be designed based on well known parameters (see, e.g., Ambion web site).

A nucleic acid molecule agent useful in the present methods also can be a co-suppressor RNA that reduces or inhibits transcription of the target histone H4 gene. A co-suppressor RNA, like an siRNA, comprises (or encodes) an RNA comprising an inverted repeat, which includes a first oligonucleotide that selectively hybridizes to the target histone H4 gene and, in operative linkage, a second oligonucleotide that is complementary and in a reverse orientation to the first oligonucleotide. In comparison to an siRNA, which comprises a functional portion of a transcribed region of the target histone gene and reduces or inhibits translation of RNA transcribed from the histone gene, a co-suppressor RNA comprises a functional portion of a transcriptional regulatory region of the target histone H4 gene (e.g., a promoter region) and reduces or inhibits transcription of the gene.

In general, the nucleotides comprising a nucleic acid molecule (e.g., a transgene) are naturally occurring deoxyribonucleotides, such as adenine, cytosine, guanine or thymine linked to 2′-deoxyribose, or ribonucleotides such as adenine, cytosine, guanine or uracil linked to ribose. However, a nucleic acid molecule also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al. (1994) Nucl. Acids Res. 22:5220-34; Jellinek et al. (1995) Biochemistry 34:11363-72; Pagratis et al. (1997) Nature Biotechnol. 15:68-73, each of which is incorporated herein by reference). Similarly, the covalent bond linking the nucleotides of a polynucleotide generally is a phosphodiester bond, but also can be, for example, a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986, 1994; Ecker and Crooke, BioTechnology 13:351360, 1995, each of which is incorporated herein by reference). The incorporation of non-naturally occurring nucleotide analogs or non-naturally occurring bonds linking the nucleotides or analogs can be particularly useful where the nucleic acid molecule (e.g., an antisense molecule or siRNA) is to be exposed to an environment that can contain a nucleolytic activity, including, for example, a cell culture medium or in a cell (e.g., a human cell), since the modified molecules can be less susceptible to degradation.

A nucleotide sequence containing naturally occurring nucleotides and phosphodiester bonds, can be chemically synthesized or can be produced using recombinant DNA methods, using an appropriate polynucleotide as a template. In comparison, a nucleotide sequence containing nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

A nucleic acid molecule encoding, for example, an antisense molecule or an siRNA, can be contained in a vector, particularly an expression vector, and can be introduced into a cell by any of a variety of methods known in the art (see, for example, Sambrook et al., “Molecular Cloning: A laboratory manual” (Cold Spring Harbor Laboratory Press 1989); Ausubel et al., “Current Protocols in Molecular Biology”, John Wiley and Sons, Baltimore, Md. (1987, and supplements through 1995), each of which is incorporated herein by reference). Such methods include, for example, transfection, lipofection, microinjection, electroporation and, with viral vectors, infection; and can include the use of liposomes, microemulsions or the like, which can facilitate introduction of the polynucleotide into the cell and can protect the polynucleotide from degradation prior to its introduction into the cell. The selection of a particular method will depend, for example, on the cell into which the polynucleotide is to be introduced, as well as whether the cell is isolated in culture, or is in a tissue or organ in culture or in situ.

For expression in a cell, a nucleic acid molecule encoding an antisense molecule, an siRNA, and the like, can be operatively linked to one or more transcriptional regulatory elements, including, for example, one or more promoters, which comprise a transcription start site; enhancers or silencers, which increase or decrease, respectively, the level of transcription of the encoded nucleic acid molecule; or terminators, which comprise a transcription stop site. Promoters and enhancers, which can be used to drive transcription can be constitutive (e.g., a viral promoter such as a cytomegalovirus promoter or an SV40 promoter), inducible (e.g., a metallothionein promoter), repressible, or tissue specific, as desired. Transcriptional regulatory element, including eukaryotic and prokaryotic promoters, terminators, enhancers, and silencers, are well known in the art and can be chemically synthesized, obtained from naturally occurring nucleic acid molecules, or purchased from commercial sources.

The present invention also relates to a method of determining whether a neoplastic disease, such as cancer, is susceptible to treatment with an agent that reduces or inhibits histone H4 activity. Such a method, which provides a tool for personalized medicine, can be practiced, for example, by detecting the level of histone H4 gene expression in at least a first neoplastic cell sample, wherein at least a about a 1.2-fold, or at least a about a 1.5-fold, or at least a about a 2-fold, or at least a about a 2.5-fold, or at least a about a three-fold increase in the level of expression of a histone H4 gene in the neoplastic cells as compared to a level the expression in corresponding normal cells indicates that the neoplastic disease is susceptible to treatment with an agent that reduces or inhibits histone H4 activity. In certain preferred embodiments the histone H4 gene is histone H4c. A cancer or other neoplastic cell sample can be a biopsy sample obtained from a cancer patient, can be cancer cells that have been adapted to culture, can be cancer cells of a panel of available cancer cells, or any other cancer cell sample.

The invention also provides methods for screening for an agent for reducing or inhibiting proliferation of neoplastic cells. The method involves measuring the ability of an agent to reduce the amount of histone H4 mRNA or histone H4 protein in a neoplastic cell. Alternatively the screening method may involve screening the ability of an agent to bind DNA or RNA of a gene encoding histone H4.

The present invention further relates to a method of treating a patient with a neoplastic disease, such as cancer. A method of the invention can be practiced, for example, by administering to the patient an agent the reduces or inhibits histone H4 activity in neoplastic cells in the patient, and can be practiced as a single therapeutic modality, or can be combined with one or more additional modalities (e.g., surgery, chemotherapy, or radiotherapy). The agent, which can be administered to the site of the neoplastic disease (e.g. the site of a cancer) or can be administered systemically, can be, for example, a nucleic acid molecule (e.g., an antisense molecule, an siRNA, a co-suppressor RNA, a ribozyme, or a triplexing agent), a peptide, a peptidomimetic, or a small organic molecule (e.g., a polyamide), which can be operatively linked to a chemotherapeutic molecule.

The present invention also provides methods for treating a patient with a neoplastic disease, such as cancer, by administering an agent that reduces or inhibits histone H4 activity. Efficacy is identified by detecting that signs or symptoms associated with the neoplastic disease are lessened. The signs and symptoms characteristic of particular types of neoplastic disease are well known to the skilled clinician, as are methods for monitoring the signs and conditions. For example, imaging methods can be used to determine that a tumor has decreased in size, or is increasing in size at a lower rate, due to treatment according to the present methods.

For administration to a patient with a neoplastic disease, including a human or other subject, the agent generally is formulated with a pharmaceutically acceptable carrier to provide a composition suitable for administration the subject. The form of the composition will depend, in part, on the route by which the composition is to be administered. Generally, the composition will be formulated such that the agent is in a solution or a suspension, such a form be suitable for administration by injection, infusion, or the like, or for aerosolization for administration by inhalation. However, the composition also can be formulated as a cream, foam, jelly, lotion, ointment, gel, or the like, or in an orally available form.

A pharmaceutically acceptable carrier useful for formulating an agent for use in a method of the invention can be aqueous or non-aqueous, for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the agent, for example, carbohydrates, such as glucose, sucrose or dextrans, antioxidants, such as ascorbic acid or glutathione, chelating agents, low molecular weight proteins or other stabilizers or excipients.

The pharmaceutical composition also can comprise an admixture with an organic or inorganic carrier or excipient, and can be compounded, for example, with the usual non-toxic, pharmaceutically acceptable carriers for tablets, pellets, capsules, suppositories, solutions, emulsions, suspensions, or other form suitable for use. The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary stabilizing, thickening or coloring agents can be used, for example a stabilizing dry agent such as triulose.

Where the agent is a nucleic acid molecule, it can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton Fla. 1984); Fraley et al., Trends Biochem. Sci., 6:77, 1981). Liposomes, for example, which consist of phospholipids or other lipids, are nontoxic, physiologically acceptable and metabolizable carriers that are relatively simple to make and administer. “Stealth” liposomes (see U.S. Pat. Nos. 5,882,679; 5,395,619; and 5,225,212) are an example of such encapsulating materials particularly useful for preparing a pharmaceutical composition.

A peptide agent useful in the present methods can contain naturally occurring amino acids and peptide bonds, or can be a modified peptide containing, for example, one or more D-amino acids in place of a corresponding L-amino acid; or one or more amino acid analogs, for example, an amino acid that has been derivatized or otherwise modified at its reactive side chain, or the peptide can be modified at its amino terminus or the carboxy terminus or both. Such peptides can have improved stability to a protease, an oxidizing agent or other reactive material the peptide may encounter in a biological environment, and, therefore, can be particularly useful in performing a method of the invention. Of course, the peptides can be modified to have decreased stability in a biological environment such that the period of time the peptide is active in the environment is reduced.

The amount of the particular agent contained in a composition can be varied, depending on the type of composition, such that the amount present is sufficient reduce or inhibit histone H4 gene expression, as appropriate, thereby treating the neoplastic disease patient. In general, an amount of an agent sufficient to provide a therapeutic benefit can be determined using routine clinical methods, including Phase I, II and III clinical trials.

The invention also provides a method of determining whether a neoplastic disease, or a neoplastic cell, is susceptible to treatment with an agent that reduces or inhibits histone H4 activity. Such a method is performed by determining that the level of histone H4 gene expression in a neoplastic cell sample for the individual is at least two-fold (e.g., 2-fold, 2.5-fold, 3-fold, or more) greater than the level of histone H4 gene expression in corresponding normal cells. In certain preferred embodiments the histone H4 gene is histone H4c. The level of histone H4 gene expression can be determined using methods as disclosed herein or otherwise known in the art. Likewise, the invention provides a method for determining whether a neoplastic disease is susceptible to treatment with an agent that reduces or inhibits activity of a histone other than H4. The method is performed by evaluating the expression of histone genes in a neoplastic cell; and where the expression of a histone gene is at least two-fold (e.g., 2-fold, 2.5-fold, 3-fold, or more) greater than the level the gene expression in corresponding normal cells; the neoplastic disease is likely to be susceptible to treatment with an agent that reduces or inhibits the expression of the histone gene.

The level of histone H4 activity can be determined by detecting the level of histone H4 gene expression. The level of histone H4 gene expression can be determined, for example, by detecting histone H4 mRNA in the cells (e.g., using oligonucleotide probes, primers, or primer pairs specific for histone H4 nucleic acid molecules), or can be determined by detecting histone H4 protein in the cells (e.g., using anti-histone H4 antibodies). One of ordinary skill in the art recognizes that there are other methods that could be used to determine levels of histone H4 gene expression, e.g. transcription assays, promoter activity reporter assays, etc.

The method may be performed in a high throughput format, thus facilitating the examination of a plurality of neoplastic cell samples, which can be the same or different or a combination thereof, in parallel. As such, the method allows for detecting the level of histone H4 gene expression in a plurality of samples, including 1, 2, 3, 4, 5, or more neoplastic cell samples or other neoplastic cell samples and, as desired, 1, 2, 3, 4, 5 or more control samples (e.g., non-neoplastic cells corresponding to the neoplastic cells). In another embodiment, the method is performed in a multiplex format, wherein the level of histone H4 gene expression is detected in at least a second neoplastic cell sample, or in at least a first corresponding normal cell sample, or in a combination thereof. Methods of performing multiplex assays in a high throughput format also are provided.

For a high throughput format, samples, which can be samples of neoplastic cells, of extracts of the neoplastic cells, or of nucleic acid molecules (e.g., RNA) isolated from the neoplastic cells, the samples can be deposited manually or robotically on a solid support (e.g., a glass slide or a silicon chip or wafer). Generally, the samples are arranged in an array or other reproducible pattern, such that each sample can be assigned an address (i.e., a position on the array), thus facilitating identification of the source of the sample. An additional advantage of arranging the samples in an array, particularly an addressable array, is that an automated system can be used for adding or removing reagents from one or more of the samples at various times, or for adding different reagents to particular samples. In addition to the convenience of examining multiple samples at the same time, such high throughput assays provide a means for examining duplicate, triplicate, or more aliquots of a single sample, thus increasing the validity of the results obtained, and for examining control samples under the same conditions as the test samples, thus providing an internal standard for comparing results from different assays.

Upon determining that a patient's neoplastic cells are amenable to treatment according to a method of the invention, the samples, which can be the same as or different from the samples examined for susceptibility to treatment, can be further examined to identify an agent useful for treating the patient. As such, the invention provides a screening assay to identify an agent useful for treating a patient with a neoplastic disease whose neoplastic cells exhibit elevated histone H4 activity. Such a method, when performed in a high throughput format, can be particularly useful for screening a library of molecules (e.g., a combinatorial library), which can, but need not, be chemically related. Methods for preparing a combinatorial library of molecules that can be tested for a desired activity are well known in the art and include, for example, methods of making a phage display library of peptides, which can be constrained peptides (see, for example, U.S. Pat. Nos. 5,622,699 and 5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference); an oligosaccharide library (York et al., Carb. Res. 285:99-128, 1996; Liang et al., Science 274:1520-1522, 1996; Ding et al., Adv. Expt. Med. Biol. 376:261-269, 1995; each of which is incorporated herein by reference); a lipoprotein library (de Kruif et al., FEBS Lett. 399:232-236, 1996, which is incorporated herein by reference); a glycoprotein or glycolipid library (Karaoglu et al., J. Cell Biol. 130:567-577, 1995, which is incorporated herein by reference); or a chemical library containing, for example, drugs or other pharmaceutical agents (Gordon et al., J. Med. Chem. 37:1385-1401, 1994; Ecker and Crooke, BioTechnology 13:351-360, 1995; each of which is incorporated herein by reference).

In related aspects the invention is based, in part, on the discovery that reducing the activity of a histone, other than histone H4, in a neoplastic cell that has elevated levels of a gene encoding the histone as compared to the levels of a corresponding normal cell, results in a reduction or inhibition of proliferation of the neoplastic cell. Thus, in certain aspects, the invention may also relate to methods and compositions for reducing or inhibiting proliferation of a neoplastic cell by reducing the activity of a histone other than histone H4. In one aspect, the invention provides a method of reducing or inhibiting proliferation of a neoplastic cell involving: (1) evaluating the expression histone genes in the neoplastic cell to identify a histone gene with expression that is elevated relative to a corresponding normal cell; (2) contacting the neoplastic cell with an agent that reduces the mRNA levels of the histone gene in the neoplasic cell, thereby reducing or inhibiting proliferation of the neoplastic cell. Thus, a neoplastic cell amenable to the methods and compositions of the invention may have an increased expression level of any gene encoding a histone as compared to the expression level of the gene in a corresponding normal cell. The gene with elevated expression levels in the neoplastic cell may be any gene that encodes a histone protein; for example, a gene that encodes histone H2A, histone H2B, histone H3 or histone H4. The agent of the above embodiment can be any agent described herein. In certain embodiments, an amenable neoplastic cell may have an increased level of histone H3.3A or H3.3B gene expression as compared to a corresponding neoplastic cell. Likewise, in certain embodiments the invention includes compositions or agents that reduce or inhibit the expression of histone H3.3A or histone H3.3B, thereby reducing or inhibiting proliferation of a neoplastic cell.

As disclosed in the examples, pyrrole-imidazole (PI) polyamide-DNA alkylator (chlorambucil) conjugates were screened for their effects on morphology and growth characteristics of human colon carcinoma cell lines, and a compound (1R-Chl; also referred to as “48R-CHL”) was identified that causes cells to arrest in the G2/M stage of the cell cycle, without any apparent cytotoxicity. Microarray analysis indicated that only one gene out of about 18,000 probed genes was significantly down regulated by the polyamide 1R-Chl, and RT-PCR and western blotting experiments confirmed that histone H4c mRNA and total H4 protein levels are reduced in treated cells (see Example 1). The nucleotide (SEQ ID NO: 1) and amino acid (SEQ ID NO:2) sequences of histone H4c are provided (see, also, GenBank Acc. No. NM_{—}003542, which is incorporated herein by reference). Down regulation of H4c mRNA by siRNA yields the same cellular response, providing target validation (Example 2). Alkylation within the coding region of the H4c gene was confirmed in cell culture. Cells treated with the pyrrole-imidazole polyamide conjugate failed to grow in soft agar, and did not form tumors in nude mice, indicating that the cells are no longer tumorigenic. These results demonstrate that histone H4 provides a target for neoplastic disease therapy in cases in which the neoplastic cells exhibit increased histone H4 activity as compared to corresponding normal cells.

The human genome contains 14 genes encoding the same H4 protein (at the level of the primary amino acid sequence); however, only the H4c gene was affected by 1R-Chl. Microarray results indicated that H4c was the most highly expressed histone H4 gene in SW620 cells, accounting for approximately 70% of total H4 mRNA. Other microarray data, which can be found on the internet at the URL—hypertext transfer protocol (“http”)://expression.gnf.org, indicates that this gene is highly expressed in various cancer cell lines, and that expression of this gene is higher in cancer cells than in normal human tissues and cell types. Indeed, histone H4c expression is far lower in normal human kidney tissue and in peripheral blood lymphocytes than in SW620 cells, accounting for less than 20% of the total H4 mRNA in kidney or lymphocytes. While sequence analysis reveals that binding sites for polyamide IR are present in all members of this gene family, only the histone H4c gene was affected by polyamide 1R-Chl (see Example 1; also referred to as “48R-CHL”). These results indicate that the high expression level and active chromatin structure of this gene marks the H4c gene as a unique polyamide target.

Down-regulation of a key component of chromatin was consistent with the observation that cells treated with 1R-Chl were blocked in the G2/M phase of the cell cycle. Cells that are unable to form their full complement of nucleosomes during DNA replication will not be able to condense their DNA into mitotic chromosomes and, therefore, will be unable to proceed through mitosis. Cell cycle arrest has also been observed by down regulation of H4 transcription in human cell lines, mediated by antisense ablation of the mRNA for the histone gene transcription factor HiNF—P, (Mitra P et al., (2003) Mol Cell Biol 23, 8110-23) similar to the presently disclosed finding that down regulation of histone H4c mRNA by 1R-Chl and H4c siRNA caused growth arrest. Surprisingly, down-regulation of histone H4 did not have a global effect on genomic transcription, since chromatin structure is thought to be central to regulation of gene expression in eukaryotic cells; however, depletion of H4 protein by a genetic approach in yeast revealed that expression of 15% of the genome was increased and expression of 10% of genes was decreased. (Kim et al. (1988) Embo J 7, 2211-9; Wyrick et al. (1999) Nature 402, 418-21). Importantly, the majority of up-regulated genes were located near yeast telomeres. Since the organization of human genes is profoundly different from that of yeast (e.g., human genome size is far greater and contains about 5-times as many genes), it is not necessarily surprising that only a small number of genes were up-regulated by 1R-Chl.

Cells treated with the polyamide failed to grow in soft agar, and did not form tumors in nude mice, indicating that polyamide-treated cells were no longer tumorigenic, and entered an irreversible cellular pathway. This compound also blocked tumorigenicity of metastatic colon carcinoma cells when administered by intravenous injection in immunocompromised mice. These results indicate that the polyamide will be active against various neoplastic cell types in which the histone H4c gene is over-expressed. Other polyamide-Chl conjugates, with different DNA sequence specificities, also may effectively block the growth of cell lines in which histone H4 expression is not up-regulated. As such, polyamide-alkylator conjugates provide a class of compounds that can be useful as human neoplastic disease chemotherapeutics.

The following examples are intended to illustrate but not limit the invention.

### EXAMPLE 1

### Inhibition of Histone H4 Gene Transcription Using a Polyamide Conjugate Inhibits Neoplastic Cell Proliferation

This example illustrates that the proliferation of neoplastic cells having elevated histone H4 levels is inhibited by reducing the histone level in the neoplastic cells. This example also demonstrates that a polyamide-Chl conjugate that blocks neoplastic cell proliferation both in vitro and in a mouse model for human colon cancer.

### METHODS

A. Polyamide Synthesis and Characterization

Polyamides were synthesized using the solid phase methods described by Baird and Dervan (Baird, et al. (1996) J. Am. Chem. Soc. 118, 6141-6146) and the identity and purity of the compounds was established by analytical HPLC and mass spectrometry analysis (MALDI-TOF-MS; see, also, U.S. Pat. No. 6,559,125). For polyamide-BODIPYL dye conjugates (Belitsky, J. M., et al. (2002), Bioorgan. Med. Chem. 10, 3313-3318), the free amine of the precursor polyamide (at the α-position of the turn amino acid) was reacted with the succinimidyl ester of BODIPYL® FL probes (Molecular Probes) and the dye conjugate was purified by HPLC. For polyamide-alkylator conjugates (Wurtz, N. R. et al. (2002) Chem. & Biol. 7:153-161) the carboxylic acid of chlorambucil (Sigma) was activated and coupled to the α-amino group of the turn amino acid R-2,4-diaminobutyric acid or S-2,4-diaminobutyric acid, and purified by HPLC. Binding affinities of the unconjugated polyamide were determined by quantitative DNase I footprinting (Trauger J W, et al. (2001) Methods Enzymol. 340:450-466) using a radiolabeled PCR product derived from the human histone H4c gene (GenBank Acc. No. NM₁₃ 003542; see, also, SEQ ID NOS:2). A 214 bp region of mRNA-coding sequence was amplified from genomic DNA from SW620 cells with PCR primers corresponding to nucleotide positions 71-90 and 265-284, and radiolabeled by the inclusion of one 5′ end-labeled primer (labeled with T4 polynucleotide kinase and γ-³²P-ATP) in the PCR reaction. Sites of alkylation in this PCR product were determined by thermal or piperidine cleavage assays (Wurtz N R, et al. (2000) Chem. & Biol. 7, 153-161). after incubation of the radiolabeled DNA with polyamide-Chl conjugates for 20 hr at 37° C. The DNA was then ethanol precipitated and dissolved in either 40 μl of 10 mM sodium citrate buffer, pH 7.2, or in 150 μl of 1 M piperidine, and incubated for 30 min at 95° C., followed by ethanol precipitation. Formic acid (0.3% for 25 min at 37° C.) was used to generate A+G sequence markers and dimethylsulfate (2% for 2 min at 23° C.) was used for the G-only reaction. (Maxam A, et al. (1980) Meth. Enzymol. 65, 497-559). Additionally, alkylation sites were mapped by primer extension using the radiolabeled top strand primer, unlabeled dNTPs and VENT polymerase (New England Biolabs) after incubation of the unlabeled PCR products with polyamides for approximately 20 hr at 37° C. and thermal cleavage as above. Footprinting and alkylation reactions were analyzed by electrophoresis on 6% sequencing polyacrylamide gels containing 8.3 M urea and 88 mM Tris-borate, pH 8.3, 2 mM EDTA. The dried gels were exposed to Kodak Bio-Max™ film with DuPont Cronex Lightning Plus™ intensifying screens at −80° C. Quantitation of the footprint titrations was by storage phosphorimage analysis utilizing Kodak Storage Phosphor™ Screens (SO 230) and a Molecular Dynamics SF Phosphorlmager™ imager. The data were analyzed using ImageQuant™ software from Molecular Dynamics.

B. Cell Culture

The human colon adenocarcinoma cell lines SW480 (American Type Culture Collection (ATCC) CCL-228), SW620 (CCL-227; derived from a lymph node metastasis from the same patient as SW480), were maintained in Leibovitz medium as recommended by the ATCC. Cell growth and morphology were monitored by phase contrast microscopy, and viability by trypan blue exclusion and an ATP assay (ApoSENSOR™ cell viability assay; BioVision). Deconvolution microscopy with polyamide-bodipy conjugates was as described. (Dudouet B, et al. (2003) Chem Biol 10, 859-67). The effects of polyamide-alkylator conjugates on cell cycle progression were monitored by FACS analysis after staining with propidium iodide (50 μg/ml). Soft agar assays were performed on cells after treatment with polyamide for 72 hr, then grown in the absence of polyamide for seven days.

C. Soft Agar Assays

Soft agar assays were performed in 6-well culture dishes using SeaPlaque low-melting temperature agarose (Cambrex Bio Science Rocklarid, Inc.). The cell growth medium was supplemented with 20% fetal bovine serum. Cells were treated for 5 days with or without polyamide, harvested by trypsin treatment, and counted using a hemocytometer. Cell viability was higher than 90% in both treated and untreated cells as determined by trypan blue exclusion assays. 3×10⁶ cells of each sample were suspended in 0.5 ml growth medium and transferred to a sterile tube containing 2 ml of a 0.375% agarose suspension in medium. Cells were gently mixed by pipetting and quickly transferred to the culture dish containing a thin layer (0.5 ml) of solidified 0.5% agarose in medium. Cultures were incubated for 1-2 weeks prior to visualization by microscopy.

D. Oligonucleotide Array Analysis

Total RNA from SW620 cells from four pooled culture wells from triplicate experiments was isolated using a Qiagen RNeasy™ Midi kit according to the manufacturer's instructions. Cells were incubated with 500 nM polyamide 1R, 1R-Chl, 1S-Chl or Chl for 72 hr prior to RNA isolation. Microarray experiments were performed at the DNA Array Core Facility of The Scripps Research Institute using Affymetrix Genechip® Human Genome U133A chips. Data were analyzed using Affymetrix MicroArray® Suite (MAS 5.0) software. RMA values for probe sets were analyzed using Significance Analysis of Microarrays (SAM) 1.21 software (Stanford University).

E. Real-Time Quantitative RT-PCR

Real-time quantitative RT-PCR analysis was performed essentially as previously described (Chuma M, et al. (2003) Hepatology 37, 198-207), using the primers described above for the H4c gene. RNA was standardized by quantification of the general housekeeping gene glyceraldehyde-3-phosphate dehydrogenase (GAPDH) using the forward primer 5′-TGCACCACCAACTGCTTAGC-3′ (SEQ ID NO:3) and the reverse primer 5′-GGCATGGACTGTGGTCATGAG-3′ (SEQ ID NO:4), as described. (Pattyn F, et al. (2003) Nucleic Acids Res 31, 122-3). Quantitative real-time RT-PCR was performed using QuantiTect™ SYBR® Green RT-PCR (Qiagen) as described. (Dudouet B, et al. (2003) Chem Biol 10, 859-67). Temperature cycling and detection of the SYBR® Green emission was performed with a Cepheid SmartCycler® system. Statistical analysis was performed on three independent quantitative RT-PCR experiments for each RNA sample.

F. Western Blot Analysis

Protein levels in polyamide-treated and untreated cells were monitored by western blot analysis using antibodies specific for histone H4 (Upstate Biotechnology) or control antibodies specific for ras (Oncogene Sciences) and p53 (Upstate). Histones were purified by acid extraction as described in the protocols provided by Upstate Biotechnology. Signals were detected by chemiluminescence after probing the blot with HRP-conjugated secondary antibody (SUPERSIGNAL West kit, Pierce). To quantify the relative levels of proteins, autoradiograms (within the linear response range of X-ray film) were converted into digital images and the signals quantified using ImageQuant™ software.

G. Ligation-Mediated PCR

Polyamides were added to approximately 2×10⁷SW620 cells and incubated in a CO₂ incubator at 37° C. for 24 hr, or subjected to DNA isolation immediately after the addition of polyamides. Genomic DNA was extracted using a Qiagen genomic extraction kit. DNA samples were digested with appropriate restriction enzymes (Dra I for H4c and Hae III for N-Ras) and subjected to thermal cleavage in 10 mM sodium citrate or in 1M piperidine (Wurtz N R, et al. (2000) Chem. Biol. 7:153-161). To generate a sequence marker, DNA (50 μg) was incubated with dimethylsulfate (0.5% for 2 min), then treated with 1 M piperidine for 30 min at 95° C. DNA samples were precipitated with ethanol twice, and used in ligation-mediated PCR with nested primers. (Garrity P A, et al. (1992) Proc. Natl. Acad. Sci. USA 89, 1021-1025). First strand synthesis was by primer extension using VENT polymerase, with a primer corresponding to nucleotide positions 44-63 on the top strand of the H4c gene (GenBank Acc. No. NM_{—}003542). The double-stranded linker sequence and linker ligation was as described. (Garrity P A, et al. (1992) Proc. Natl. Acad. Sci. USA 89, 1021-1025). 35 cycles of PCR (using the H4c primer and one linker primer) was followed by primer extension with a radiolabeled primer, corresponding to nucleotide positions 71-90 on the top strand of the H4c gene or to positions 88822 to 88842 on the top strand of the N-Ras gene. The final radiolabeled DNA was analyzed on a sequencing gel.

H. Animal Experiments

CD-1 nu/nu mice were purchased from The Scripps Research Institute Division of Animal Resources, and experimental protocols were approved by the Scripps Institutional Animal Welfare Committee. Mice were injected in one flank with 1×10⁷ SW620 cells, and tumors were allowed to develop for 28 days prior to euthanasia. Polyamides were dissolved in PBS and injected into the tail vein in a total volume of 200 μl as described herein.

### RESULTS

Blocking Neoplastic Cell Proliferation with a Polyamide-Chlorambucil Conjugate

A highly tumorigenic human colon carcinoma cell line SW620 that was derived from a lymph node metastasis (Leibovitz A, et al. (1976) Cancer Res 36, 4562-9) was used for these experiments. Five different hairpin polyamides conjugated at the α-position of the hairpin turn amino acid with the nitrogen mustard chlorambucil (Chl) were synthesized (Dudouet B, et al. (2003) Chem. Biol 10, 859-67; Wurtz N R, et al. (2000) Chem. Biol. 7, 153-61; Baird E E, et al. (1996) J. Am. Chem. Soc. 118, 6141-46). Each of these molecules had a different DNA sequence specificity afforded by the polyamide amino acid sequence (polyamides 1R-Chl to 5-Chl, Table 1) (Dervan, P B (2001) Bioorgan. Med. Chem. 9, 22 15-2235; Dervan P B et al. (2003) Cur. Opin. Struct. Biol. 13, 284-99) and would be expected to alkylate adenine and guanine bases in the minor groove located adjacent to the polyamide recognition site (Dudouet B, et al. (2003) Chem. Biol 10, 859-67; Wurtz N R, et al. (2000) Chem. Biol. 7, 153-61; Baird E E, et al. (1996) J. Am. Chem. Soc. 118, 6141-46). Chemical synthesis utilized solid phase methods (Baird E E et al. (1996) J. Am. Chem. Soc. 118, 6141-46) and the purity and identity of all compounds was established by analytical HPLC and mass spectrometry (MALDI-TOF-MS). FIG. 1 shows the structures of two such polyamides (1R/S-Chl). Footprinting experiments with non-alkylating, hydrolyzed derivatives of the conjugates (replacing the chlorambucil chlorides with hydroxyls) demonstrated no loss in DNA binding affinity or specificity with the Chl derivatives compared to the parent hairpin polyamides. (Wurtz N R et al. (2000) Chem. & Biol. 7, 153-61).

### Table 1

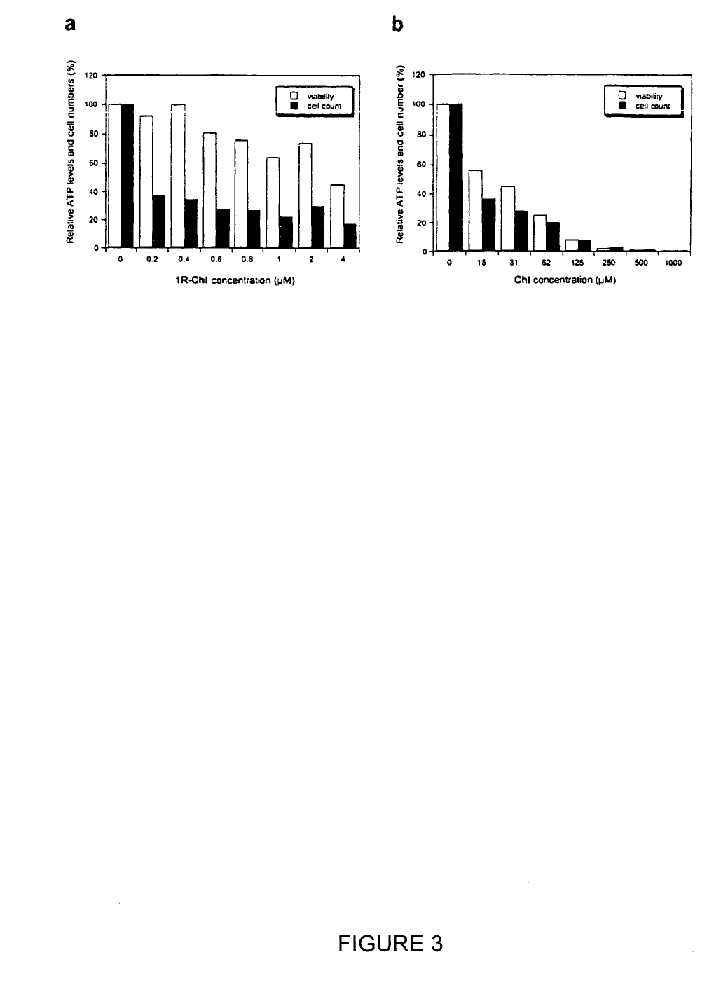

TABLE 1
Growth arrest and cell morphology change requires the sequence-specificity of 1 R-CHl. Polyamide structures are
shown schematically (as in FIG. 1), along with the predicted DNA target site for each polyamide [13], where W = A or
T. Pairing of an Im opposite a Py targets a C.G base pair. The Py/Py, β/Py and β/β pairs are degenerate and target
both A.T and T.A base pairs. Both the hairpin turn amino acid and the terminal β-Dp recognize A.T or T.A base pairs.
With the exception of 1S-Chl, all polyamides were synthesized with R-2,4-dimaminobutyric acid as the turn amino
acid. Morphology change was assessed, and growth arrest is based on cell counts. Viability was assessed by trypan
blue exclusion and an ATP assay (see Supplementary FIG. 3).

| Polyamide | | Sequence | Cell morphology change | Growth arrest | Viability |
|---|---|---|---|---|---|
| 18-Chl | 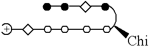 | 5′-WGGWGW-3′ | yes | yes | + |
| 2-Chl | 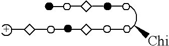 | 5′-WGCWGWW-3′ | no | no | + |
| 3-Chl | 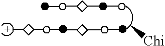 | 5′-WGCWGCW-3′ | no | no | + |
| 4-Chl | 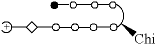 | 5′-WGWWWW-3′ | | cytotoxic | |
| 5-Chl | 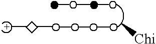 | 5′-WGWGWW-3′ | | cytotoxic | |
| 6-Chl | 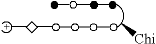 | 5′-WGWGGW-3′ | no | slight | + |
| 7-Chl | 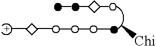 | 5′-WGGWCW-3′ | no | slight | + |
| 15-Chl | 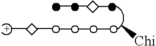 | 5′-WGWGGW-3′ | no | no | + |
| 1R | 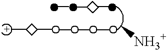 | 5′-WGGWGW-3′ | no | no | + |

Chemotherapeutic molecules, such as DNA alkylators, topoisomerase inhibitors or histone deacetylase inhibitors, alter the morphology and growth characteristics of cancer cells in culture. A series of polyamide conjugates, with different DNA sequence specificities, was screened for their effects on the morphology of SW620 colon carcinoma cells in culture. Among the polyamides tested (Table 1), microscopic inspection demonstrated that only 1R-Chl altered the morphology of the cells (Not shown). Untreated SW620 cells were typically either round or spindle shaped, while incubation with polyamide 1R-Chl altered the morphology of these cells, wherein the cells were enlarged, flattened, and irregular. The morphological change was apparent after 48 hours and was induced with as low a concentration as 250 nM. Cells treated with this compound failed to divide and consequently, fewer cells were seen compared to the untreated cells. However, cells treated with this polyamide are viable (>90% by trypan blue exclusion) and metabolically active (assessed by ATP levels; FIG. 3. Thus, 1R-Chl is a cytostatic, rather than a cytotoxic agent in this cell line. Neither 1R lacking Chl, nor different polyamide-Chl conjugates (e.g., 2-Chl, ImPy-βImPy-(R-2,4-Daba^{Chl})-PyPyβImPy-βDp and those shown in Table 1, above) altered the morphology or growth properties of these cells (at 0.5 μM). Chl (at 0.5 μM) was without effect on SW620 cell morphology or growth, but was cytotoxic at higher concentrations (FIG. 3). The two polyamide-conjugates that had the lowest sequence specificity of the tested compounds, 4-Chl and 5-Chl, were highly cytotoxic and were not studied further. These compounds are similar to conventional alkylators that target vast numbers of sites in the genome. Binding sites for 1R-Chl are expected to be of the type 5′-WGGWGW-3′ (SEQ ID NO:5; where W=A or T, Table 1), while S enantiomer 1S-Chl is expected to bind DNA in the reverse orientation as the R enantiomer (5′-WGWGGW-3′; SEQ ID NO:6). (Melander C et al. (2000) Chemistry 6, 4487-97). After identification of 1R-Chl, the structural requirements for growth arrest were probed with a second series of compounds (Table 1). Altering DNA the sequence specificity by either scrambling the sequence of pyrrole and imidazole rings (6-Chl and 7-Chl) or by inverting the chirality of the turn amino acid (1S-Chl, FIG. 1) abolished the morphological change and growth arrest observed with 1R-Chl. Furthermore, the parent polyamide 1R, lacking Chl, was inactive. These results demonstrate that the morphological change and cytostatic properties of 1R-Chl require both the alkylating moiety, chlorambucil, and the sequence-specific DNA-binding moiety of the polyamide suggesting that the change in cell morphology and growth are due to sequence-specific DNA alkylation and concomitant gene silencing.

To test cell permeability and nuclear localization of the polyamides in the SW620 cell line, fluorescent analogues were synthesized of active polyamide 1R-Chl and inactive polyamide 1S-Chl, Similar to the Chl conjugates, fluorescent BODIPYL® FL dye was coupled to the α-amino group of the turn amino acid¹⁵ (FIG. 1). As observed using deconvolution microscopy, both 1R-bobipy and 1S-bodipy entered the nucleus of live, unfixed human colon cancer cells. Given the similar structures of the bodipy and chlorambucil conjugates (FIG. 1), these results indicated that the chlorambucil derivatives also should be cell permeable and should localize in the nucleus (see below).

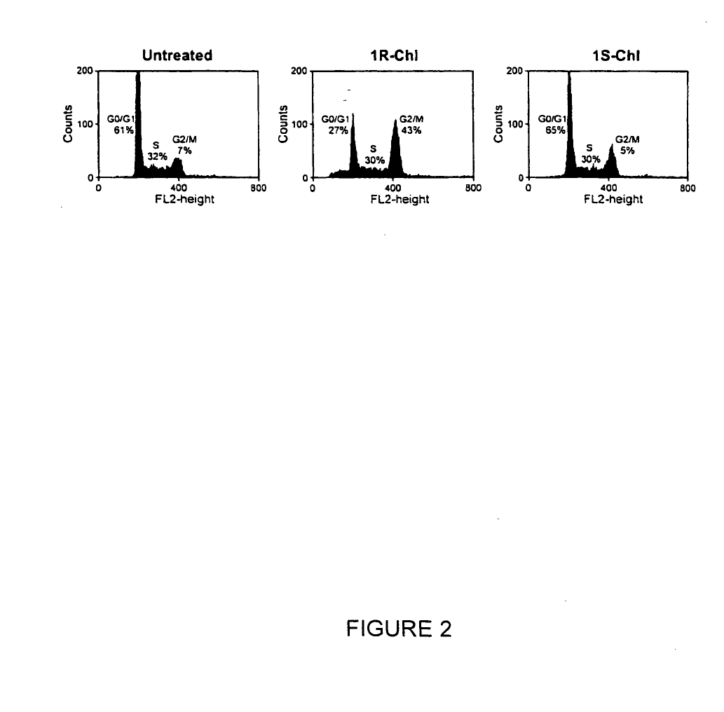

To assess the mechanism of action of polyamide 1R-Chl, the DNA content of untreated, 1R-Chl and 1S-Chl-treated SW620 cells was monitored by fluorescence activated cell-sorting (FACS) analysis after propidium iodide staining (FIG. 2B). The DNA profiles of untreated and 1S-Chl-treated cells were similar, with approximately 5% to 7% of the cells in G2/M (4C DNA content); however, treatment with polyamide 1R-Chl caused an increase in the fraction of cells with a 4C DNA content to 43%. This result indicates that polyamide 1R-Chl arrests cells in the G2/M phase of the cell cycle. A small fraction of the 1R-Chl-treated cells were apoptotic, as evidenced by less than a 2C DNA content. A time course experiment revealed that cell cycle arrest occurred approximately two days after polyamide treatment, similar to the time required to observe the change in cell morphology described above. On longer exposure to the polyamide, increasing numbers of cells had a 4C DNA content, consistent with a block in the cell cycle at G2/M.

Gene Target of the Polyamide

The effects of polyamide treatment on genomic transcription were monitored by DNA microarray analysis using Affymetrix high-density U133A arrays, which contain oligonucleotides representing approximately 18,000 human genes. SW620 cells were treated (in triplicate) with no polyamide, or with polyamides 1R-Chl, 1S-Chl, and chlorambucil at a concentration of 0.5 μM in culture medium for 72 hr, which is a sufficient time for growth arrest (see above). Total RNA was isolated, converted into fluorescent cRNA and hybridized to the oligonucleotide microarrays. While Chl affected the transcription of a large number of genes, the levels of transcription of a surprisingly limited number of genes were affected by polyamide-Chl treatment (77 genes up regulated and 35 down regulated for 1R-Chl).

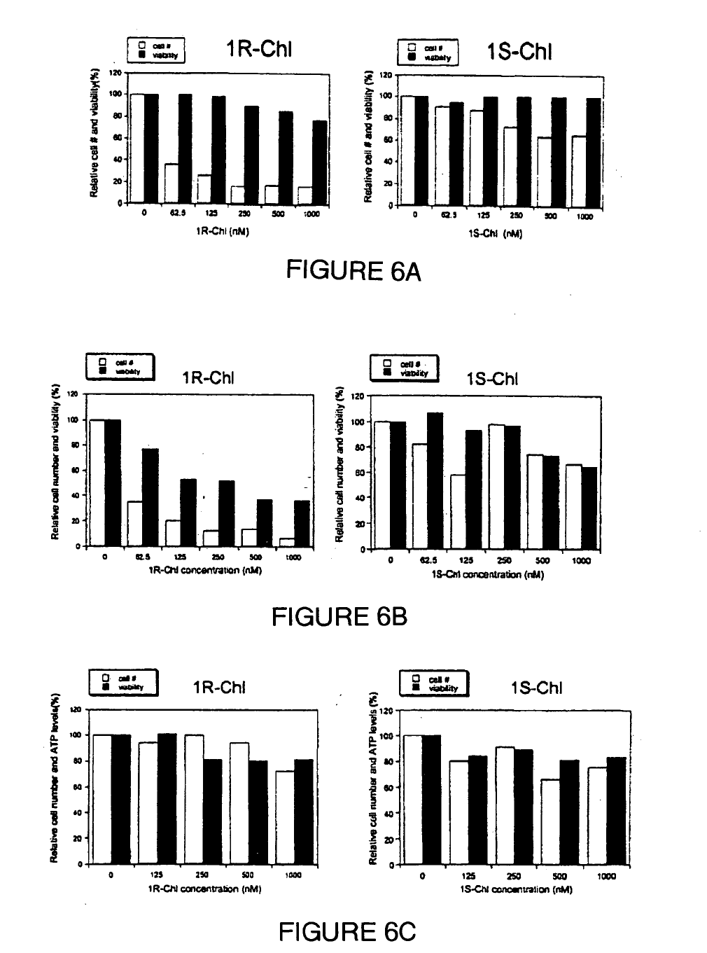

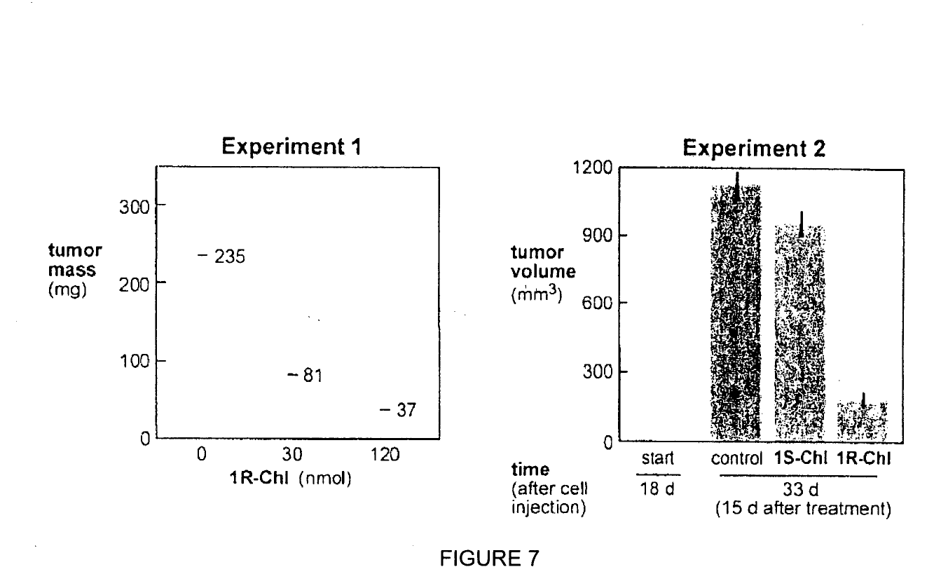

Of the genes that were affected by 1R-Chl, 23 genes were uniquely down regulated (Table 2) and 70 genes were uniquely up regulated when compared to 1R, Chl and 1S-Chl. Of the specifically up-regulated genes, only three were increased in expression by 2-fold or more. These genes encode β-tubulin (GenBank accession number NM_{—}001069), epithelial membrane protein 1 (NM_{—}001423) and CD24 antigen (NM_{—}013230). Only one gene (GenBank Acc. No. NM_{—}003542) was uniquely down-regulated by 1R-Chl by a threshold value of at least 2-fold. This gene encodes histone H4 family member G (H4c gene), a member of the gene family that encodes histone proteins of the nucleosome. Down regulation of histone H4 could reasonably account for the growth effects observed with 1R-Chl. Although Affymetrix U133A chips contain oligonucleotides representing all members of the H4 gene family, only histone H4C transcription was affected. H4c was the most abundantly transcribed H4 gene in SW620 cells, accounting for approximately 70% of total H4 mRNA (FIG. 6). H4c mRNA was also elevated relative to normal cell in certain other cell lines (FIG. 7). Real time quantitative reverse transcriptase PCR verified that this gene was down-regulated approximately 2-fold by 1R-Chl.

### Table 2

TABLE 2
Affymetrix genechip analysis of genes that are uniquely down regulated by
polyamide 1R-Chl, and are not affected by polyamide 1S-Chl or by Chl.

| | Affymetrix | Relative | Fold | Genbank | |
|---|---|---|---|---|---|
| | Probe | Expression | Change | ID | Name |
| 1 | 205967_at | 0.49 | −2.03 | NM_003542 | Histone 1, H4c |
| 2 | 207060_at | 0.62 | −1.62 | NM_001427 | Engrailed homolog 2 |
| 3 | 203998_s_at | 0.62 | −1.61 | NM_005639 | Synaptotagmin 1 |
| 4 | 209478_at | 0.65 | −1.54 | NM_144998 | Stimulated by retinoic acid 13 |
| 5 | 201626_at | 0.65 | −1.53 | BE300521 | Insulin induced gene 1 |
| 6 | 201195_s_at | 0.65 | −1.53 | NM_003486 | Solute carrier family 7 |
| 7 | 212708_at | 0.66 | −1.52 | AL049450 | Hypothetical protein LOC339287 |
| 8 | 209146_at | 0.66 | −1.52 | BC010653 | Sterol-C4-methyl oxidase-like |
| 9 | 204798_at | 0.67 | −1.49 | NM_005375 | v-myb myeloblastosis viral oncogene homolog |
| 10 | 202294_at | 0.68 | −1.47 | BC064699 | Propionyl Coenzyme A carboxylase, beta |
| | | | | | polypeptide |
| 11 | 205542_at | 0.68 | −1.46 | NM_012449 | Six transmembrane epithelial antigen of the |
| | | | | | prostate |
| 12 | 205258_at | 0.69 | −1.46 | NM_002193 | Inhibin, beta B (activin AB beta polypeptide) |
| 13 | 218281_at | 0.69 | −1.45 | NM_016055 | Mitochondrial ribosomal protein L48 |
| 14 | 218579_s_at | 0.70 | −1.43 | NM_021931 | DEAH (Asp-Glu-Ala-His) box polypeptide 35 |
| 15 | 205199_at | 0.70 | −1.43 | NM_001216 | Carbonic anhydrase IX |
| 16 | 218005_at | 0.70 | −1.42 | BC041139 | Zinc finger protein 22 (KOX 15) |
| 17 | 203860_at | 0.71 | −1.42 | NM_000282 | Propionyl coenzyme A carboxylase, alpha |
| | | | | | polypeptide |
| 18 | 209303_at | 0.71 | −1.41 | NM_002495 | NADH dehydrogenase (ubiquinone) Fe—S |
| | | | | | protein 4 |
| 19 | 203614_at | 0.71 | −1.41 | NM_021645 | KIAA0266 gene product |
| 20 | 222258_s_at | 0.72 | −1.39 | NM_014521 | SH3-domain binding protein 4 |
| 21 | 221908_at | 0.73 | −1.37 | AK094682 | Hypothetical protein FLJ14627 |
| 22 | 216384_x_at | 0.74 | −1.36 | AF170294 | Prothymosin a14 mRNA |
| 23 | 218421_at | 0.75 | −1.33 | NM_022766 | Ceramide kinase |

The level of histone H4 protein was examined in polyamide-treated and control SW620 cells by western blot analysis with an antibody to H4 (data not shown). 1R-Chl treatment of cells for 72 hr resulted in about a 50% to 70% reduction in H4 protein level, consistent with the relative contribution of the H4c gene to total H4 histone mRNA synthesis. As controls, the protein levels of Ras and p53 (not shown) were monitored in polyamide-treated and control cells and found no differences. The polyamides were without effect on the transcription of these genes in either microarray or RT-PCR experiments. These results indicate that the change in cellular morphology and growth inhibition of the tumor cells was due to the inhibition of H4c transcription by polyamide 1R-Chl.

DNA Binding Properties of the Polyamides

The DNA binding and alkylation properties of polyamides 1R and 1S, and their Chl conjugates with a DNA fragment derived from the of the human H4c gene (isolated by PCR amplification from genomic DNA) were explored. DNase I footprint analysis was used to monitor the binding specificities and affinities of the parent compounds lacking Chl. Previous studies with a polyamide-Chl conjugate (where the Chl chlorines were replaced with hydroxyls) demonstrated no loss in binding affinities compared to the parent polyamide lacking Chl. Although the H4c gene contains four match sites for polyamide 1R (5′-WGGWGW-3′; SEQ ID NO:5; see Table 3), only two of these sites were occupied in the footprinting experiment (with K_{d}s of 0.3 and 0.7 nM). The two additional match sites are purine tracts, a sequence type that is often poorly bound by hairpin polyamides.

### Table 3

TABLE 3
Polyamide 1R binding sites in the histone H4c gene: in intro assays.
Binding affinities were determined by quantitative DNase I footprinting
(with polyamide 1R), and alkylation by thermal cleavage (with polyamide
1R-Chl) and the radiolabeled H4c PCR product. The alkylated nucleotides
are shown in red.

| | 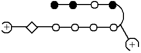 | 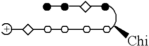 |
|---|---|---|
| Sequence | K_{d} (nM) | Alkylation |
| 5′-tgaggagact-3′ | >100 | No |
| 3′-actcctctga-5′ | | |
| 5′-cgaggtgtgc-3′ | 0.7 | Yes |
| 3′-gctccacacg-5′ | | |
| 5′-cgtcacctat-3′ | 0.3 | No |
| 3′-gcagtggata-5′ | | |
| 5′-ggtctcctga-3′ | >100 | No |
| 3′-ccagaggact-5′ | | |

A previous study demonstrated that a polyamide with the turn amino acid R-2,4-dimaminobutyric acid binds its match site with 170-fold higher affinity than the corresponding S enantiomer [30], documented the importance of the chirality of the hairpin turn in determining binding affinity. Although potential “match” sites for polyamide 1S are present in this PCR product, 1S failed to bind this DNA fragment at polyamide concentrations up to 100 nM. Since alkylation of the template strand of the H4c gene is most likely to lead to transcriptional inhibition (Oyoshi T et al. (2003) J Am Chem Soc 125, 4752-4), thermal cleavage assays were used to monitor site-specific alkylation by the polyamide-Chl conjugates on the bottom strand of the H4c PCR product. Polyamide 1R-Chl alkylated one site in this DNA, corresponding to one of the two match sites described above. Alkylation at the second high affinity site observed in the footprinting experiment may be prevented by local DNA microstructure and/or by the orientation of Chl at the turn amino acid. Close inspection of the sequencing gel revealed that alkylation occurred at two nucleotides, the guanine located two bases downstream from the polyamide binding-site and at the adenine located proximal to the turn amino acid. Consistent with the binding experiment, 1S-Chl yielded only minor alkylation products, even at the highest polyamide concentration tested (100 nM). Phosphorimage analysis indicated that 1S-Chl exhibited an approximately 100-fold lower alkylation efficiency than 1R-Chl at this site. No alkylation events were observed with either 1R-Chl or 1S-Chl on the top strand of this PCR product, and polyamides 4-Chl and 5-Chl also fail to significantly alkylate the H4c PCR product on either strand. One additional binding site for polyamide 1R is present in the promoter element of the H4c gene (5′-TGGTGA-3′, located 95 bp upstream from the transcription start site); however, 1R-Chl fails to alkylate this site in vitro (data not shown).

Ligation-mediated PCR was used to monitor alkylation of the H4c gene in genomic DNA of SW620 cells, and strong alkylation was observed in cellular chromatin (FIG. 8C, lane 5). Only the G residue, two bases downstream from the polyamide binding-site appeared to be alkylated in the cell nucleus. In contrast to the in vitro experiment, no alklyation was detected with 1S-Chl in cell culture (lane 6). In a control experiment, the DNA isolation protocol was initiated immediately after addition of 1R-Chl to the cells. No alkylation was observed under these conditions (lane 4), indicating that alkylation did not occur during DNA isolation and purification. These results demonstrate the direct binding and alkylation of the H4c gene by 1R-Chl in living SW620 cells.

Experiments were next performed to determine whether polyamide 1R-Chl alkylates potential target sites in a gene whose transcription is not affected by this compound in SW620 cells. Since 1R-Chl had no effect on N-Ras gene expression (as determined in the microarray experiment and confirmed by RTPCR) or N-Ras protein levels, the experiments focused on the coding region of this gene. LM-PCR experiments demonstrated that both 1R-Chl and 1S-Chl alkylate sites near the 5′ end of this gene in isolated genomic DNA in vitro (on the coding strand), but these sites were not available for alkylation in the cell nucleus. It is likely that differences in chromatin organization between the H4c and N-Ras genes account for differential polyamide accessibility and the ability of 1R-Chi to regulate expression of these genes.

Effects of 1R-Chl on Cell Lines of Different Origin

To assess the generality of the effects of 1R-Chl, growth rates and viability for various other human cell lines were monitored in the absence or presence of conjugates 1R-Chl and 1S-Chl. Polyamide 1S-Chl was without effect on the morphology, viability or growth of any cell lines tested. In contrast, 1R-Chl showed variable effects in the different lines (Table 4). Based on these results, these cell lines were be divided into three groups: (1) two lines where the compound had no effects up to 1 μM concentration (Hep3B hepatocellular carcinoma cells and 293 embryonic kidney cells); (2) three cell lines in which 1R-Chl was growth inhibitory and cytotoxic (22Rv1 prostate, MiaPaCal pancreatic and HeLa cervical carcinoma); and, (3) those that responded similarly to SW620 cells, where the growth characteristics of the cells were altered, without apparent cytotoxicity (as assessed by measuring ATP levels). These latter cell lines included SW420, the lymphoblast cell line K562, and SaOS2 osteosarcoma cells (Table 4). FIG. 9A-C shows representative results for one cell line in each class. FACS analysis revealed that 1R-Chl had no effect on cell cycle progression in the two unaffected cell lines. In contrast, 1R-Chl blocked cell cycle progression (G2/M arrest) in two out of three cell lines where the compound was growth inhibitory and cytotoxic, and caused G2/1M arrest in each of the cell lines where the compound was cytostatic (Table 4).

TABLE 4

| | | | | G2/M | | Effect of 1R- |
|---|---|---|---|---|---|---|
| | | Growth | | cell cycle | Relative H4c | Chl on H4c |
| Cell line | Origin | inhibition | Viability^{a} | arrest^{b} | mRNA^{c} | mRNA^{d} |
| Unaffected | kidney | − | + | − | 1.13 ± 0.02 | 0.86 ± 0.01 |
| 293 | | | | | | |
| Hep3B | Liver | − | + | − | 1.16 ± 0.28 | 1.17 ± 0.28 |
| Cytotoxic | prostate | + | − | − | 0.50 ± 0.04 | 0.36 ± 0.03 |
| 22Rv1 | | | | | | |
| MiaPaCal | pancreas | + | − | + | 0.12 ± 0.01 | 0.62 ± 0.06 |
| HeLa | cervix | + | − | + | 0.25 ± 0.02 | 0.66 ± 0.04 |
| Cytostatic | colon | + | + | + | 1.00 | 0.38 ± 0.02 |
| SW620 | | | | | | |
| SW480 | colon | + | + | + | 0.38 ± 0.02 | 0.44 ± 0.02 |
| K562 | CML | + | + | + | 0.54 ± 0.05 | 0.23 ± 0.02 |
| SaOs2 | bone | + | + | + | ND | ND |

^{a}All assays were performed on cells treated with 1R-Chl at 0.5 μM for 4 days. ATP levels measured with the ApoSensor assay. “+” indicates ATP levels comparable to untreated cells, while ”−” indicates ATP levels below 40% of untreated cells.
^{b}As determined by FACS analysis.
^{c}H4c mRNA levels (±standard deviation) in untreated cells relative to GAPDH mRNA, normalized to the H4c/GAPDH ratio for SW620 cells, as determined by qRT-PCR.
^{d}The values shown represent the ratio of H4c mRNA in the 1R-Chl treated cells to untreated cells, normalized for GAPDH levels, and are the averages of three determinations (±standard deviation).
^{e}Not determined.

The effect of 1R-Chl on cell morphology was then evaluated in additional cell-lines. These experiments, using light-microscopy techniques, demonstrated that 1R-Chl at a concentration of 500 nM (4-6 incubation days at 37° C.) also changed the morphology and decreased the cell number in the following cell lines: HeLa, Calu-1, K562, SK BR 3, MIA CaPa-2, 22Rv1, SW480, and MCF-7 cells (see Table 5). In these cell lines, treatment with 1R-Chl (500 nM) resulted in the cells becoming enlarged and/or irregular in shape as compared to untreated control cells or those treated with 500 nM of 1S-Chl. The Saos2, Molt-4 (studies in Molt-4 were conducted at a concentration of 25 nM of 1R-Chl), and Capan-1 cell lines showed a decreased number of cells when they were treated with 500 nM of 1R-Chl as compared to cells that were untreated or treated with same concentration of 1S-Chl; however, in these cell lines there was no significant alteration of cell morphology observed in response to 1R-Chl treatment. Thus of the cell-lines investigated only two, Hep3B and 293, were not visually affected by 1R-Chl.

### Table 5

TABLE 5
lists the cell lines, their origin and growth conditions
used in the studies described below.

| Cell Line (ATCC | | |
|---|---|---|
| Number) | Type of Cancer | Growth Medium |
| SW620 (CCL-227) | Colorectal | Leibowitz' Medium |
| | adenocarcinoma | |
| HeLa (CCL-2) | Cervical | Dulbecco's Modified |
| | adenocarcinoma | Eagle Medium |
| Calu-1 (HTB-54) | Lung | Iscove's Modified |
| | epidermocarcinoma | Dulbecco's Medium |
| K562 (CCL-243) | Chronic myelogenous | RPMI Medium |
| | leukemia | |
| SK BR 3 (HTB-30) | Mammary gland | Dulbecco's Modified |
| | adenocarcinoma | Eagle Medium |
| MIA CaPa-2 (CRL- | Pancreatic | Dulbecco's Modified |
| 1420) | carcinoma | Eagle Medium |
| 22Rv1 (CRL-2505) | Prostatic | RPMI Medium |
| | carcinoma | |
| SW480 (CCL-228) | Colorectal | DMEM:RPMI (1:1) |
| | adenocarcinoma | |
| Saos2 (HTB-85) | Osteosarcoma | Dulbecco's Modified |
| | | Eagle Medium |
| Molt-4 (CRL-1582) | Acute lymphoblastic | RPMI Medium |
| | leukemia | |
| Capan-1 (HTB-79) | Hepatopancreatico | Iscove's Modified |
| | adenocarcinoma | Eagle Medium |
| MCF-7 (HTB-22) | Mammary gland | Dulbecco's Modified |
| | adenocarcinoma | Eagle Medium |
| Hep3B (HB-8064) | Hepatocellular | Dulbecco's Modified |
| | carcinoma | Eagle Medium |
| 293 (CRL-1573) | Kidney/adenovirus | Dulbecco's Modified |
| | 5 DNA | Eagle Medium |

The effect of 1R-Chl on cell proliferation was also further studied in experiments with additional cell lines. For these experiments cells were cultured with either no polyamide or with 62.5, 125, 250, 500, and 1000 nM of 1R-Chl or 1S-Chl, incubated for 4 to 6 days at 37° C., and collected for manual counting. The results of the experiments showed that, in addition to its effect on SW620, 1R-Chl was also able to decrease the cell number in HeLa, Calu-1, K562, SK BR 3, MIA CaPa-2, 22Rv1, SW480, Saos2, Molt-4, Capan-1, and MCF-7, whereas 1S-Chl had no significant effect in any of the cell lines screened. In cell lines such as MIA Capa-2 and 22Rv1, the cell number was decreased to less than 50% at a 1R-Chl dose as low as 62.5 nM, but in general all twelve cell lines showed a cell count between 5 to 40% when the 1R-Chl dose was 500 or 1000 nM. In contrast, Hep3B, and 293, cells were resistant to the anti-proliferative effects of 1R-Chl. In these three cell lines, the relative cell count at any 1R-Chl dose was comparable to that of either untreated cells (0 nM) or cells treated with equivalent doses of 1S-Chl. The viability of the cells was monitored by the ApoSENSOR™ assay, and it was found that neither 1R-Chl nor 1S-Chl significantly compromised the viability of any cell lines at the dose range studied, except for Molt-4, in which 1R-Chl, but not 1S-Chl, was toxic at a dose as low as 25 nM.

The results of experiments described above demonstrated that SW620 cells were arrested at the G2/M boundary of the cell cycle when treated with 1R-Chl, but not with 1S-Chl. Thus the effect of 1R-Chl on the cell cycle of the other cell lines was also investigated. The results of these experiments demonstrated that the cell lines which were responsive to 1R-Chl treatment showed a tendency to increase the population of cells arrested at the G2/M periphery when treated with 500 nM of 1R-Chl, but not when untreated or treated with 500 nM of 1S-Chl (1S-Chl lane). The cell lines that were not responsive to the 1R-Chl treatment expectedly showed no significant alteration in their cell cycle profile when treated with either 500 nM of 1R-Chl, equivalent amount of 1S-Chl or not treated. The increase in the number of G2/M cells treated with 1R- or 1S-Chl relative to the number of untreated G2/M cells was calculated, and plotted in a graph (not shown). According to these numbers, the cell lines were categorized into two groups. The “G2/M arrest tendency” group contained cell lines whose 1R-Chl-treated cell number present at G2/M was increased by as much as 10 to 40 percentile points relative to the untreated cells, whereas the “non-arrested” group contains the cell lines that only showed a relative increase of less than 10 percentile points. The “G2/M arrest tendency” group of cells includes SW620, HeLa, Calu-1, K562, SK-BR 3, MIA CaPa-2, 22Rv1, SW480, MCF-7, Saos2, Molt-4, and Capan-1 cells and the “non-arrested” group includes Hep3B and 293 cells. No cell line showed a significant change in G2/M population when treated with 1S-Chl relative to the untreated cells.

The effects of 1R-Chl on H4c mRNA levels in representative cell lines were also monitored. Polyamide 1R-Chl reduced the level of H4c mRNA in each of the cell lines where growth inhibition was observed, but was without effect on H4c mRNA levels in the two unaffected cell lines 293 and Hep3B (Table 4). No correlation was found between the relative levels of H4c mRNA in untreated cells (relative to either the GAPDH control mRNA) and the effects of 1R-Chl on cell proliferation (Table 4). These results were obtained by qRT-PCR and confirmed by northern blotting (not shown).

Tumorigenicity of SW620 Cells Treated with Polyamide 1R-Chl

A standard soft-agar assay was employed to assess the potential tumorigenicity of polyamide 1R-Chl-treated versus untreated cells. Equal numbers of untreated and polyamide-treated cells were inoculated into soft agar (without polyamide), and grown for up to two weeks. Untreated cells, and cells treated with control compounds (1S-Chl, 1R lacking Chl, and Chl alone) formed colonies, whereas cells pre-treated with 1R-Chl failed to grow, although trypan blue exclusion indicated that the cells were viable. This result indicates that 1R-Chl induces the cells to reverts to an irreversible non-tumorigenic phenotype. Moreover, and similar to the morphological change observed in standard cell culture conditions, growth arrest required both a specific polyamide DNA-binding domain and the chlorambucil (Chl) alkylating moiety.

In Vivo Evaluation of 1R-Chl.

To determine whether the loss of anchorage-dependent growth reflects a loss of tumorigenicity in vivo, animal studies were performed in athymic nude mice (CD-1 nu/nu mice). Groups of 5 nude mice were injected with 1×10⁷SW620 cells that were either untreated (group 1), or treated for three days prior to injection with 500 nM polyamide 1R-Chl (group 2) or 1S-Chl (group 3). Each of the mice in groups 1 and 3 developed tumors measuring about 1-1.5 cm in diameter after 23 days, while none of the mice in group 2 developed tumors. As a more stringent test of the efficacy of this compound as a potential human therapeutic for colon cancer, groups of 5 nude mice were injected with SW620 cells. After one week, when tumors began to form, mice were injected intravenously with 200 μl of either PBS, or 30 or 120 nanomoles of polyamide 1R-Chl (in PBS), followed by a second injection three days later. After 28 days, the animals were euthanized and tumors were dissected and weighed (FIG. 10B). Polyamide treatment substantially suppressed tumor growth, in a dose-dependent manner. Mice that were injected with 30 or 120 nmoles of 1R-Chl had tumors that weighed an average of 35% and 16%, respectively, that of the tumors of control mice. In a second experiment, mice were again injected with SW620 cells, tumors were allowed to establish and tumor volumes were determined prior to polyamide treatment and at 15 days post treatment. Three doses of polyamide 1R-Chl (120 nmoles, administered on treatment days 0, 2,4) prevented any significant increase in tumor volume, whereas mice receiving a similar dosing regimen of polyamide 1S-Chl developed tumors comparable to the untreated control animals (FIG. 7, Experiment 2). Importantly, no obvious toxicity was associated with polyamide treatment in vivo at a polyamide concentration and dosing regimen where the therapeutic result was obtained.

The basic in vivo pharmacokinetic properties of the 1R-Chl were also examined. For these studies, a 30 nmol dose of polyamide was injected into the tail vein of pairs of normal Balb/c mice (2 mg/kg). This dose was found effective in preventing tumor growth in colon cancer xenografts in athymic nu/nu mice as described above. Mice were sacrificed at various time intervals post injection and the blood serum concentration of polyamide determined by LC-MS by The Scripps Research Institute's (TSRI) Core Mass Spectrometry Facility. This method used an extraction method for blood serum and followed by a sensitive MS detection and quantification protocol. Briefly, blood was allowed to coagulate at room temperature for 24 hours, and the sample was centrifuged at 3100 rpm for 10 min to separate serum from the clot. A 2 μL aliquot of a control polyamide (of different molecular mass, at 100 pmol/μL) was added as an internal standard to 200 μL of serum. To this, 800 μL of chilled methanol was added, the sample was vortexed, incubated on ice for 15 min, and then centrifuged at 12000 rpm for 10 min. The supernatant was harvested and concentrated to 50 μL for analysis by electrospray mass spectrometry. For liquid chromatography, a 50 mm×2 mm C18 column was used with an Agilent® 1100 Liquid Chromatography system. Polyamide standards from 100 to 12500 fmol/μL in serum were used and the concentration of samples was determined by with an Agilent® 1100 single quadrapole ElectroSpray Mass Spectrometer. For the anti-cancer compound 1R-CHL, it was found that serum polyamide concentration reached a maximum of 15 μg/mL (10 μM) at 1 hour post-injection and the half-life of the compound was 2.8 hours, corresponding to a constant of elimination (kel) of 0.27 h-1. These are very favorable PK values and are comparable to those of N-acetyl-p-aminophenol (acetomenophen, Tylenol). Based on these values, a volume distribution of 19.7 L/70 kg (corresponding to a human body mass) could be calculated, based on an average weight of 23 gm/mouse in this experiment. This volume distribution is indicative of a hydrophilic compound with good blood and body distribution, comparable in these parameters to the FDA-approved aminoglycoside antibiotic gentamicin.

### EXAMPLE 2

### Inhibition of Histone H4 Gene Transcription Using siRNA Inhibits Neoplastic Cell Proliferation

This example extends the results of Example 1 by demonstrating that an siRNA specific for the histone H4 gene inhibits proliferation of tumor cells that express elevated levels of histone H4. In this example, certain actions of H4c siRNA are also compared to actions of 1R-Chl. Where applicable all methods used in Example 1 also apply in this example.

To validate that histone H4c was the gene target responsible for growth arrest of SW620 cells, cells were transfected with siRNAs specific for H4c or for the general housekeeping gene, GAPDH. Three synthetic double stranded siRNAs targeting different sequences in the histone H4C gene were obtained in HPLC-purified and annealed form from Ambion, Inc. The sense sequences were as follows:

| | |
|---|---|
| HIST1H4C-1: | |
| 5′-GGGCAUUACAAAACCGGCUtt-3′ | (SEQ ID NO:7) |
| HIST1H4C-2: | |
| 5′-GGUGUGCUUAAGGUUUUCUtt-3′ | (SEQ ID NO:8) |
| HIST1H4C-3: | |
| 5′-GCGCAUUUCCGGUCUUAUCtt-3′ | (SEQ ID NO:9) |

Cells were transfected with 25-100 nM siRNA using the Silencer™ siRNA Transfection Kit (Ambion, Inc.). Cell growth and morphology change were evaluated 72 or 96 hr after transfection by counting cells in a hemocytometer, and by visually inspecting cells in a phase contrast microscope.

Transfection of SW620 cells with HIST1H4C-1 (SEQ ID NO:7), but not with HIST1H4C-2 (SEQ ID NO:8) or HIST1H4C-3 (SEQ ID NO:9), caused a morphological change that was comparable to that induced by treatment with polyamide 48R-CHL. Cell numbers also were decreased in HIST1H4C-1 (SEQ ID NO:7) transfected cell cultures, indicating that cell proliferation was blocked as was observed with 48R-Chl (see Table 6). To further validate H4c as the gene target responsible for growth arrest of SW620 cells, SW620 cells were transfected with siRNA to H4c (HIST1H4C-1; SEQ ID NO:7) or to the general housekeeping gene glyceraldehyde3-phosphate dehydrogenase (GAPDH), or with a scrambled sequence siRNA. Cells transfected with GAPDH siRNA showed decreased levels of GAPDH protein but no change in phenotype and only mild effects on growth (not shown). The scrambled sequence siRNA was without effect on cell morphology or growth. In contrast, transfection of H4c siRNA (HIST1 H4C-1; SEQ ID NO:7) under the same conditions caused the same morphology change and similar growth arrest as observed with 1R-Chl. H4c siRNA (HIST1H4C-1; SEQ ID NO:7) caused a 73 (+1-5) % reduction in cell number after 3 days, whereas 1R-Chl caused an 85 (+1-5) % decrease in cell number over the same time period, relative to the untreated cells. Quantitative RT-PCR confirmed that this siRNA (HIST1H4C-1; SEQ ID NO:7) down regulated H4c mRNA ˜8.6-fold compared to the control, scrambled sequence siRNA. In other experiments, it was foune that 2-fold down regulation of H4c transcription by siRNA (HIST1H4C-1; SEQ ID NO:7) was sufficient to cause growth arrest in SW620 cells. Thus it appears that inhibition of H4c transcription is responsible, at least in part, for the observed change in cellular morphology and growth with 1R-Chl.

### Table 6

TABLE 6
Effect of H4c siRNA on SW620 cell growth.

| | Untreated | Hist1H4C-1 siRNA | 48R-Ch1 (1 μM) |
|---|---|---|---|
| Relative cell number | 100% | 37% | 14% |

These results demonstrate that transfection with an H4c siRNA (HIST1H4C-1; SEQ ID NO:7) caused the same morphology change and growth arrest as was observed with 1R-Chl (Example 1), and confirm that inhibition of H4c transcription is responsible for the observed change in cellular morphology and growth of tumor cells.

Effects of Polyamide 1R-Chl on Nuclear Structure

SW620 cells were examined by Hoechst staining and fluorescence microscopy after incubation with IR- or 1S-Chl (0.5 μM) for 72 hours to assess the effects of 1R-Chl on nuclear structure. 1R-Chl, but not the inactive polyamide 1S-Chl, caused the nucleus to enlarge relative to untreated cells. Since previous studies have documented that polyamides targeted to satellite DNA can cause chromatin opening, experiments were performed to determine whether the change in nuclear size observed with 1R-Chl is due to polyamide binding at numerous sites in genomic DNA or to a reduction in H4 protein. SW620 cells were transfected with H4c siRNA (HIST1H4C-1; SEQ ID NO:7) or with the scrambled sequence siRNA, with the result that only the H4c siRNA (HIST1H4C-l; SEQ ID NO:7) caused a similar enlargement of the cell nucleus as 1R-Chl, indicating that a loss of H4 protein leads to chromatin decondensation and enlargement of the cell nucleus.

Effects of 1R-Chl and HIST1H4C-1 (SEQ ID NO:7) on v-Myb Expression.

Experiments described above demonstrated that polyamide 1R-Chl specifically down regulated 23 genes in the SW620 colon cancer cell line. Of the 23 genes, and in addition to H4c (described above) the gene v-Myb was decreased by ˜1.5 fold. Accordingly, further experiments were performed to evaluate possible roles of v-Myb in the mechanism of 1R-Chl action. Experiments using qRT-PCR demonstrated that siRNA to H4c (HIST1H4C-1; SEQ ID NO:7) also down regulated v-Myb transcription. However, other siRNAs (control siRNA of scrambled sequence, an siRNA to GAPDH or to another member of the H4 gene family) had no effect on v-Myb expression, as detected by qRT-PCR. As expected, siRNA to v-Myb down regulated v-Myb, but not H4c or GAPDH. Prior studies in the literature have established that v-Myb is a target in cancer therapy, and down regulation of v-Myb with siRNA does lead to a loss of proliferation in cell culture experiments in our laboratory. Taken together, these data suggest that down regulation of histone H4c transcription by either siRNA or chemically with 1R-Chl leads to down regulation of v-Myb, which in turn could be partly or wholly responsible for growth inhibition with 1R-Chl.

Effects of Reducing by siRNA the Expression of a Histone H4 Gene That is not Elevated Relative to a Corresponding Normal Cell.

The above described experimental results indicate that histone H4c is over expressed in cancer cells, relative to normal cells. This finding comes from microarray data in SW620 cells comparing these results to data that is available on the internet for various cancer cell lines and normal human cells and tissues (www.gnf.org). Based on these findings, it was predicted that an siRNA to a histone H4 gene that is not highly expressed in SW620 cells would not have the antiproliferative properties of 1R-Chl or siRNA to the histone H4c gene. To evaluate this hypothesis, the effect of decreasing the mRNA of H4i by siRNA in SW620 cells was investigated. This gene is expressed at less than 10% of the level of H4c and accounts for less than 10% of the total histone H4 mRNA in SW620 cells. The histone H4i gene is expressed at comparable levels in normal cells and tissues compared to cancer cells. The siRNA to H4i down regulated histone H4i mRNA levels in the SW620 cells but did not have any effect on cell growth, morphology or cell cycle progression of the cells, showing that siRNAs to this histone gene that is not highly expressed histone in SW620 cells is not effective in reducing or inhibiting proliferation of the cells. Thus, it is likely that the methods and compositions of the invention are most effective when histone genes that are highly expressed as compared to normal cells are targeted by the agents of the invention.

### EXAMPLE 3

### Targeting Histone Genes in Addition to Histone H4 for Reducing or Inhibiting Proliferation of Neoplastic Cells.

Nucleosomes are composed of four core histones, H2A, H2B, H3 and H4, encoded by multiple histone genes in the human genome. One expectation then was that siRNAs for the other histone mRNAs that are over expressed in cancer cells should have the same effect on cell morphology, growth and cell cycle progression as the siRNA to H4c and 1R-Chl. Inspection of the microarray data for SW620 cells indicated that two genes for histone H3 were highly expressed relative to the other genes encoding histone H3. These genes were histone H3.3A and histone H3.3B. Accordingly, siRNAs to the mRNAs encoded by these genes were generated and their effects on cell morphology, growth and cell cycle progression of SW620 cells were investigated. The siRNAs to these histone H3.3A and histone H3.3B genes selectively reduced the mRNA levels of the respective histone genes, these siRNAs were found to have the same effects on SW620 cell morphology, growth and cell cycle as the siRNA to H4c and as 1R-Chl. Thus, down regulation of genes encoding histone proteins that are over expressed in neoplastic cells as compared to normal cells is an effective strategy for inhibition of the growth of neoplastic cells, without affecting corresponding non-neoplastic cells.

Although the invention has been described with reference to the above examples, it will be understood that modifications and variations are encompassed within the spirit and scope of the invention. Accordingly, the invention is limited only by the following claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method of reducing or inhibiting proliferation of neoplastic cells, comprising contacting the neoplastic cells with an agent that binds to a histone H4 gene or to RNA encoding histone H4, whereby histone H4 activity is reduced or inhibited, thereby reducing or inhibiting proliferation of the neoplastic cells.

2. The method of claim 1, wherein the neoplastic cells are cancer cells.

3. The method of claim 1, wherein the neoplastic cells are selected from the group consisting of colon carcinoma cells, hepatocellular carcinoma cells, cervical carcinoma cells, lung epidermocarcinoma cells, mammary gland adenocarcinoma cells, pancreatic carcinoma cells, prostatic carcinoma cells, osteosarcoma cells, melanoma cells, acute promyelocytic leukemia cells, acute lymphoblastic leukemia cells, hepatocancreatico adenocarcinoma cells and Burkitt's lymphoma B cells.

4. The method of claim 1, wherein the gene encoding histone H4 is histone H4c.

5. The method of claim 1, wherein said agent reduces the level of histone H4 mRNA or histone H4 protein in said cell.

6. The method of claim 1, wherein said agent comprises a pyrrole-imidazole polyamide.

7. The method of claim 6, wherein said pyrrole-imidazole polyamide is operatively linked to a chemotherapeutic molecule.

8. The method of claim 6, wherein said pyrrole-imidazole polyamide binds DNA having the sequence 5′-WGGWGW-3′.

9. The method of claim 6, wherein said pyrrole-imidazole polyamide is operatively linked to a alkylator.

10. The method of claim 7, wherein said chemotherapeutic molecule is chlorambucil.

11. The method of claim 1, wherein said agent is 1R-Chl.

12. The method of claim 1, wherein said agent comprises a nucleic acid molecule.

13. The method of claim 12, wherein said nucleic acid comprises an antisense molecule, a small interfering RNA (siRNA), a co-suppressor RNA, a ribozyme, or a triplexing agent.

14. The method of claim 13, wherein the nucleic acid molecule comprises a siRNA comprising a nucleotide sequence as set forth in SEQ ID NO:7.

15. A composition for reducing or inhibiting proliferation of neoplastic cells, said composition comprising a DNA or RNA binding domain operatively linked to a chemotherapeutic molecule, wherein said DNA or RNA binding domain binds to DNA or RNA corresponding to a gene encoding histone H4;
or a pharmaceutically acceptable salt or complex thereof.

16. The composition of claim 15, wherein said gene encoding histone H4 is histone H4c.

17. The composition of claim 15, wherein said DNA binding domain is binds to the sequence 5′-WGGWGW-3′.

18. The composition of claim 15, wherein said DNA binding domain comprises a pyrrole-imidazole polyamide.

19. The composition of claim 15, wherein said chemotherapeutic molecule is an alkylator.

20. The composition of claim 15, wherein said compound is 1R-Chl.

21. A method of determining whether neoplastic cells are susceptible to treatment with an agent that reduces or inhibits histone activity, comprising detecting the expression level of a histone gene in a sample of said neoplastic cells; and detecting the expression level of a histone gene in a non-neoplastic control cell sample that corresponds to the cell type of said neoplastic cells;
wherein at least a three-fold increase in the expression level of said histone gene in the neoplastic cells as compared to expression level of said histone gene in corresponding normal cells indicates that the neoplastic disease is susceptible to treatment with an agent that reduces or inhibits histone activity.

22. The method of claim 21, wherein the level of histone gene expression is determined by detecting histone H4 mRNA in the cells.

23. The method of claim 21, wherein the level of histone H4 gene expression is determined by detecting histone H4 protein in the cells.

24. The method of claim 21, wherein said neoplastic cells are cancer cells.


SEQUENCE LISTING (10 sequences)

SEQ ID NO: 1 - DNA, 390 bp, Homo sapiens
atgtctggtcgcggcaaaggcggaaaaggcttggggaagggtggtgctaagcgccatcgtaaggtgctccgggataacatccagggcattacaaaaccggctattcgccgtttggctcggcgcggtggcgtcaagcgcatttccggtcttatctatgaggagactcgaggtgtgcttaaggttttcttagagaacgttattcgagacgccgtcacctatacggagcacgccaagcgcaaaactgtcacagccatggatgtagtatatgccctaaaacgtcaggggcgcactctgtatggcttcggcggctgaatctaagaatacgcggtctcctgagaacttcaaaaaacaaaaacaaaaaaacccaaaggcccttttcagggccgctca

SEQ ID NO: 2 - protein, 103 aa, Homo sapiens [sequence omitted]

SEQ ID NO: 3 - DNA, 20 bp, Artificial Sequence
  Description of Artificial Sequence Synthetic amplification primer
tgcaccaccaactgcttagc

SEQ ID NO: 4 - DNA, 21 bp, Artificial Sequence
  Description of Artificial Sequence Synthetic amplification primer
ggcatggactgtggtcatgag

SEQ ID NO: 5 - DNA, 6 bp, Artificial Sequence
  Description of Artificial Sequence Synthetic polyamide binding site
wggwgw

SEQ ID NO: 6 - DNA, 6 bp, Artificial Sequence
  Description of Artificial Sequence Synthetic polyamide binding site
wgwggw

SEQ ID NO: 7 - DNA, 21 bp, Artificial Sequence
  Description of Combined DNA/RNA Molecule Synthetic small interfering RNA
gggcauuacaaaaccggcutt

SEQ ID NO: 8 - DNA, 21 bp, Artificial Sequence
  Description of Combined DNA/RNA Molecule Synthetic small interfering RNA
ggugugcuuaagguuuucutt

SEQ ID NO: 9 - DNA, 21 bp, Artificial Sequence
  Description of Combined DNA/RNA Molecule Synthetic small interfering RNA
gcgcauuuccggucuuauctt

SEQ ID NO: 10 - DNA, 34 bp, Homo sapiens
atgaggagactcgaggtgtgcttaaggttttctt